(19) United States
(12) Patent Application Publication
(10) Pub. No.: US 2026/0221241 A1
(43) Pub. Date: Jul. 30, 2026

(54) Using Artificial Intelligence / Machine Vision for Automated Attendance Logging and Real Time Service Delivery Documentation, and Systems and Methods Therefor

(71) Applicant: Therap Services, LLC, Torrington, CT (US)

(72) Inventors: David Lawrence Turock, Fort Lauderdale, FL (US); Justin Mark Brockie, Wolcott, CT (US); Mohammad Jahangir Alam, Rocky Hill, CT (US); James Michael Kelly, Morris, CT (US); Minar Mahmud, Dhaka (BD); Md Habibur Rahman, Dhaka (BD); Richard Allen Robbins, Lenox, MA (US); Tanvir Shahriar Rifat, Dhaka (BD); Seemanta Ahmed Shubho, Dhaka (BD); Md Al Zihad, Dhaka (BD); James Joseph Brockman, Millstone Township, NJ (US); Khandker Mohammed Nurul Afsar, Narayanganj (BD); Muhammad Atiqur Rahman Imon, Cumilla (BD); Nazmus Sadat, Dhaka (BD)

(73) Assignee: Therap Services, LLC, Torrington, CT (US)

(21) Appl. No.: 19/358,511

(22) Filed: Oct. 15, 2025

Related U.S. Application Data
(60) Provisional application No. 63/751,354, filed on Jan. 30, 2025.

(51) Int. Cl. G16H 10/60; G06F 21/62; G16H 30/20; G16H 40/20; H04L 9/32

(52) U.S. Cl. CPC G16H 10/60; G06F 21/6209; G16H 40/20; H04L 9/3236; G06F 2221/2141

(57) ABSTRACT

Methods and systems for electronically recording the presence of an individual at a location, including individuals under care by a caregiver, include HIPAA-compliant receipt and recording of appearance data of an individual, comparing the data to stored records, and if matched, storing an attendance record. A sensor captures images of an individual at a point within the location, the image is hashed and compared with hashes in a database by a computer system, and if matched, an electronic attendance record, which indicates that the individual is present at the point, is created and stored in an attendance database. The electronic attendance record may be real time service delivery documentation, may include service and program information, and may be an electronic visit verification.

### CROSS-REFERENCE TO RELATED APPLICATIONS

This application claims, and is entitled to claim, a right of priority to U.S. Provisional Patent Application Ser. No. 63/751,354 filed Jan. 30, 2025 (the '354 Application), and is entitled to the benefit of the filing date thereof.

This application incorporates the entirety of the following U.S. Patents:
U.S. Pat. No. 12,217,316 filed as U.S. patent application Ser. No. 17/827,521 on May 27, 2022 (“the '316 Patent”);
U.S. Pat. No. 11,915,806, filed as U.S. patent application Ser. No. 17/941,329 on Sep. 9, 2022 (“the '806 Patent”);
U.S. Pat. No. 11,475,983 filed as U.S. patent application Ser. No. 16/695,591, on Nov. 25, 2019 (“the '983 Patent”);
U.S. Pat. No. 8,281,370 filed as U.S. patent application Ser. No. 11/604,577, on Nov. 27, 2006 (“the '370 Patent”); and
U.S. patent application Ser. No. 19/222,011 filed on May 29, 2025 (“the '011 Application”).

The '316 Patent is a continuation-in-part of U.S. Pat. No. 11,449,954 filed as U.S. Patent Application Ser. No. 16/750,388 on Jan. 23, 2020, which is a continuation-in-part of U.S. Pat. No. 10,586,290 filed as U.S. patent application Ser. No. 15/197,120 on Jun. 29, 2016, which is a continuation-in-part of U.S. patent application Ser. No. 13/675,440 (“the '440 Application”) filed Nov. 13, 2012. The '316 Patent is also a continuation-in-part of U.S. Pat. No. 11/410,759 filed as U.S. patent application Ser. No. 16/811,429 on Mar. 3, 2020, which is a continuation of U.S. Pat. No. 10,622,103 filed as U.S. patent application Ser. No. 15/636,826 on Jun. 6, 2017.

The '806 patent is a continuation of the '983 Patent, which claims priority to the '440 Application, which is a continuation-in-part of U.S. Patents Nos. 8,615,790 and 8,813,054, both of which are divisions of the '370 Patent.

All description, drawings, and teachings set forth in the '316, '806, '983, and '370 Patents and the '354 Application and '011 Applications are expressly incorporated by reference herein.

### BACKGROUND OF THE INVENTION

Traditional methods rely on manual input or checklists. These methods may not be sufficient to comply with federal and state regulations such as HIPAA and the Cares Act, GDPR, PIPEDA, and as further described in the '983 Patent at 2:64-3:5, and the '316 Patent at Col. 3, Lines 21-35 and Col. 4, Lines 41-50 (collectively “HIPAA-type regulations”).

On Dec. 13, 2016, an act entitled “An Act to accelerate the discovery, development, and delivery of 21st century cures, and for other purposes,” which was signed into law as Pub. L. 114-255 and commonly referred to as the “21st Century Cures Act,” is a further HIPAA-type regulation. Section 12006 of this law added section (1)(5)(A) to 42 U.S.C. § 1396b which reads in relevant part as follows:
The term “electronic visit verification system” means, with respect to personal care services or home health care services, a system under which visits conducted as part of such services are electronically verified with respect to (i) the type of service performed; (ii) the individual receiving the service; (iii) the date of the service; (iv) the location of service delivery; (v) the individual providing the service; and (vi) the time the service begins and ends.

The '806 Patent, which the present application incorporates by reference, discloses systems and methods for electronic verification of service visits as defined in Section 12006 of the 21st Century Cures Act. See '806 Patent at 16:41-53 (“identification information about the staff involved in service delivery”); 48:13-47; 61:1-7, 21-44, (type of service performed, i.e. “Service Description”; “identify the individual” receiving the service; “Data Collection Date”; “location” of service delivery; “Begin Time and End Time”); see also id. FIGS. 12-13, 17, 22, 30, 34, 35.

### SUMMARY OF THE INVENTION

The Automated Attendance System leverages camera-based facial recognition technology to streamline attendance tracking. Unlike traditional methods that rely on manual input or checklists, this system automatically registers individuals as they enter or exit a location. Configurable cameras capture and analyze facial data using a privacy-focused hashing mechanism, ensuring that facial images cannot be reversed and reconstructed. This method creates a unique, secure facial pattern for future identification.

By requiring physical presence for attendance logging, the system reduces fraud risks, ensuring only authorized individuals are recorded. The hashing process is akin to password encryption, which protects the integrity of the facial data. It can accommodate various hardware options, from advanced smart cameras to everyday devices like smartphones, offering a cost-effective solution.

Integrated service authorization checks enable real-time verification against attendance records and allow for automatic adjustments when necessary. Complex cases are flagged for manual review, ensuring that human intervention can resolve discrepancies. This seamless automation minimizes human error and enhances operational efficiency.

Embodiments of this invention may have applications for analysis of attendance in a location by devices, sensors and machines either in addition to or in conjunction with human staff, guardians or others and may have the ability to confirm information provided by sensors or computers in compliance with HIPAA-type regulations, as well as security procedures and objectives of furthering person-centered care.

Methods and systems for electronically recording the presence of an individual at a location, including individuals under care by a caregiver, include HIPAA-compliant receipt and recording of appearance data of an individual, comparing the data to stored records, and if matched, storing an attendance record. A sensor captures images of an individual at a point within the location, the image is hashed and compared with hashes in a database by a computer system, and if matched, an electronic attendance record, which indicates that the individual is present at the point, is created and stored in an attendance database. The electronic attendance record may be real time service delivery documentation, may include service and program information, and may be an electronic visit verification.

This disclosed invention is directed to the challenges of accurately recording the attendance of individuals at a location, and to specific improvements in acquiring and managing attendance data that address these challenges. The system and method disclosed as embodiments of the invention improve the acquisition, processing, and storing attendance data at a location. Systems and devices that perform the functions at least of: (1) acquiring facial image data of an individual; (2) creating a hash of the image data; and (3) comparing that hash to a database containing previously stored hash data, (4) recording the time of arrival of an individual and the time that individual departed, (5) computing the duration that a given individual was in attendance, and (6) computing the concomitant intersection in time of proper subsets of individuals identities were and are neither routine, well-understood, nor conventional in the field of attendance monitoring.

### BRIEF DESCRIPTION OF DRAWINGS

FIG. 1 illustrates the overall system and its infrastructure.

FIG. 1A illustrates the architecture and the interaction between the main software suites, the application, and the physical sensors.

FIG. 1B illustrates the API and user experience for a demo of an automated attendance system.

FIG. 2 illustrates the Admin interface with the Attendance Device option and “New” link for creating a device.

FIG. 2A illustrates the General section of the Admin interface where the Attendance Device creation process begins.

FIG. 2B illustrates the General section of the Admin interface where the Attendance Device creation process begins.

FIG. 2C illustrates the Attendance Device creation form with fields for device name and sensor/device ID for the check-in device.

FIG. 2D illustrates the Attendance Device List in the Admin interface.

FIG. 2E illustrates the Attendance Device details form with existing information before any edits.

FIG. 2F illustrates the Attendance Device details form in editable mode for making changes.

FIG. 2G illustrates the updated Attendance Device form after changes have been saved.

FIG. 3 illustrates how a Conference Session is created in the Admin interface and linked to “In” and “Out” attendance devices for automated tracking.

FIG. 3A illustrates the Admin interface with the Conference Session option and “New” link for creating a session

FIG. 3B illustrates the Conference Session creation form with fields for session name, program name, and assigning “In” and “Out” device

FIG. 4 illustrates how to intake attendees.

FIG. 4A illustrates the process which begins from the mobile app dashboard by navigating to the IDF tab.

FIG. 4B illustrates the screen with the Create New button.

FIG. 4C illustrates a form where the user inputs the attendee's first name, last name, and date of birth before submission.

FIG. 4D depicts the interface where camera icons are used to initiate capturing photos of the attendee's face.

FIG. 4E illustrates how previously captured photos may be retaken by selecting them again, if necessary.

FIG. 4F illustrates the form submission after the photos are taken.

FIG. 4G illustrates the Program field that is used to select the Program for the Conference Session.

FIG. 4H illustrates that attendee enrollment is confirmed after clicking the Submit button.

FIG. 4I illustrates a message indicating successful mapping to the Program.

FIG. 4J illustrates that the user needs to click on the IDF tab on the mobile app dashboard

FIG. 4K illustrates the user needs to click on the Take Photos button.

FIG. 4L illustrates the selection of attendees for whom no photo has been taken.

FIG. 4M depicts a method where a user selects the Camera icons to begin capturing a minimum of three facial photos of an attendee.

FIG. 4N illustrates a method for retaking photos that have been marked with an error icon after the system identifies faces other than the attendee's.

FIG. 4O depicts the user selecting the Submit button to finalize the photo-taking process after the images have been captured.

FIG. 4P illustrates the user selecting the IDF tab on the mobile application dashboard.

FIG. 4Q illustrates an interface that includes the Enroll Program button.

FIG. 4R depicts the Program field used to select a conference session for enrolling the attendee.

FIG. 4S illustrates the user selecting the attendee to be enrolled from the Individual field.

FIG. 4T illustrates the user selecting the Submit button to enroll the attendee into the selected program.

FIG. 4U illustrates a confirmation message indicating that the attendee has been successfully mapped to the program.

FIG. 5 illustrates the flow of Conference Attendance.

FIG. 5A illustrates the process which starts from the Admin tab by navigating to the List link beside the Conference Session option in the General section.

FIG. 5B illustrates the ‘Conference Session Search’ list and the draft Conference Session from listed.

FIG. 5C illustrates that the draft ‘Conference Session’ form may be seen where Attendance may be recorded for the session once the Start button is clicked.

FIG. 5D illustrates that multiple Conference Sessions cannot be started simultaneously. In the event of an on-going session, its termination is required which may be achieved by clicking on the End button on the respective ‘Conference Session’ form

FIG. 5E illustrates that once a ‘Conference Session’ begins, the form status changes to ‘Started’ allowing users to track the check-in and check-out by clicking on the Details button.

FIG. 5F illustrates that users are able to see if attendees have checked-in and checked-out on the ‘Conference Session Details’ page.

FIG. 5G illustrates that once the Conference Session is started, the check-in camera automatically records the attendees as they walk in before the camera.

FIG. 5H illustrates that after checking-in, attendees checking-out from the conference preferably appear before the check-out camera for at least 3 seconds for their check-out to be successfully recorded.

FIG. 5I illustrates the first step to view Attendance.

FIG. 5J illustrates the parameters required to search Attendance Data.

FIG. 5K illustrates that the attendees, whose check-in and check-out has been completed, are shown on the Attendance grid.

FIG. 6A depicts the Session List for the Real Time Service Data module.

FIG. 6B depicts a connector box labeled ‘A’ which highlights the Create Session button.

FIG. 6C, which follows the flow from connector ‘A’ in the previous FIG. 6B, depicts that the system configured to three fields need to be filled in order for a user to Create Session.

FIG. 6D illustrates the Session Summary 605.

FIG. 6E illustrates the Session Summary 605 after the submission of the Attendee List 606.

FIG. 6F depicts a connector box labeled ‘B’ which highlights the Check-In 608 button.

FIG. 6G, which follows the flow from connector ‘B’ in the previous FIG. 6F, depicts the system comprises Face Recognition 607 to Check-In and Check-Out from a session.

FIG. 6H illustrates a connector box labeled ‘C’ which highlights the registration of Face Recognition 607.

FIG. 6I illustrates the pop-up for data submission, which follows the flow from connector ‘C’in the previous FIG. 6H.

FIG. 6J depicts a connector box labeled ‘D’which highlights the Finish 610 button.

FIG. 6K, which follows the flow from connector ‘D’ in the previous FIG. 6J, illustrates the process to Finish Session 611.

FIG. 7A illustrates the initial user interface for the Real Time Service Data Search 701 feature, presenting a date selection field ‘MM/DD/YYYY’ 702 with an associated calendar widget, and an initial ‘No Data Available’ 703 message prompting the user to perform a search.

FIG. 7B illustrates the Real Time Service Data Search interface after a successful search.

FIG. 7C illustrates the user interacting with the data grid by opening the ‘Program’ dropdown 714, which displays a list of available programs for selection within a service data entry.

FIG. 7D illustrates the user interacting with the ‘Description Code’ dropdown 715, which now displays a context-sensitive list of relevant codes.

FIG. 7E illustrates the data grid after ‘Program’ and ‘Description Code’ have been selected for the entries, with a connector box ‘E’.

FIG. 7F, following connector ‘E’ from FIG. 7E, illustrates the ‘Select Service’ 718 pop-up window, which enables the user to specify service details.

FIG. 7G illustrates the data grid after the ‘Select Service’ 718 pop-up has been completed, showing the ‘Service’ 719 column is now populated for each row with detailed service information.

FIG. 7H depicts a connector box ‘B’ highlighting the ‘Generate Data’ 720 button, which indicates the final action for the user is to click this button to finalize and create the attendance records.

FIG. 7I, following connector ‘B’ from FIG. 7H, illustrates the ‘Attendance Records’ success confirmation pop-up 721/722 that displays the newly processed ‘Attendance Form ID’ 723 and ‘Service Form ID’ 724 with a ‘CREATED’ 725 status after the ‘Generate Data’ 720 button is clicked.

FIG. 7J illustrates the final outcome on the main ‘Attendance’ 726 dashboard, where the newly generated attendance record for ‘Black, Gerald’ 727 is visible in the grid, confirming the data conversion and storage, with a ‘Time In/Out’ 728 tooltip showing the captured session times.

### DETAILED DESCRIPTION OF THE INVENTION

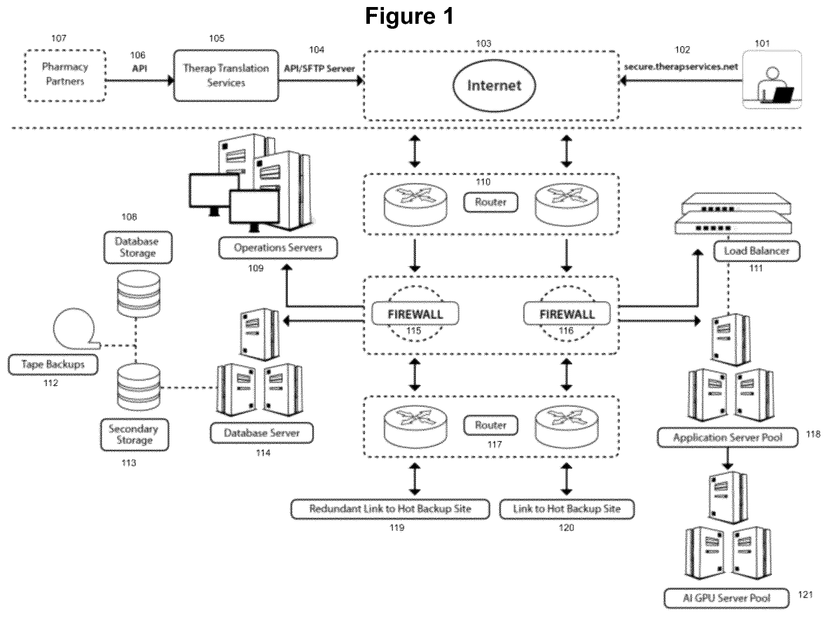

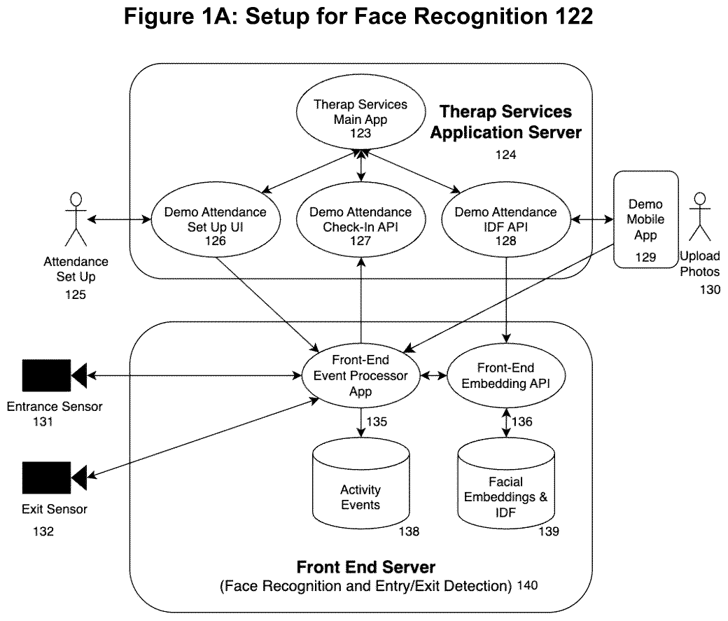

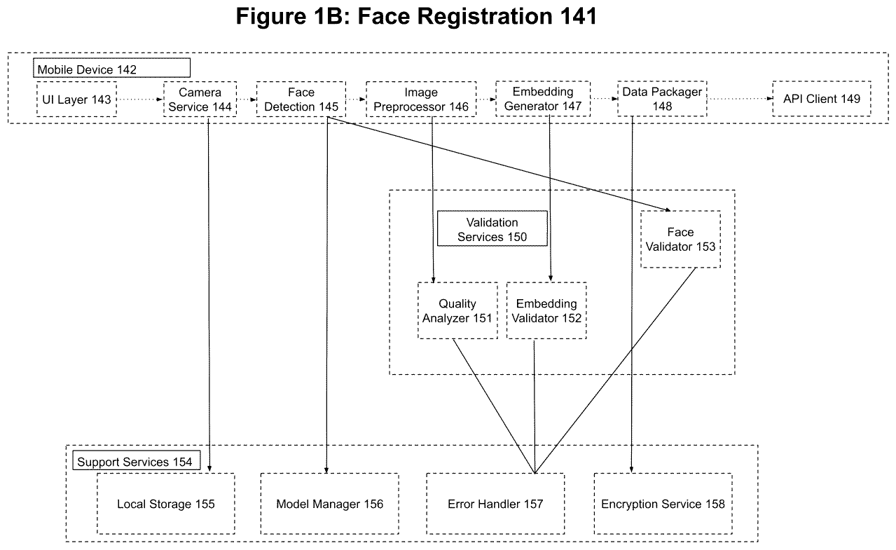

FIG. 1 illustrates the overall system and its infrastructure. The infrastructure manages data flow from various sources through a structured and secure path and is compliant with HIPAA-type regulations. Users 101 connect to the system via the Internet 103 using the secure. therapservices. net 102, while Pharmacy Partners 107 send data through an API 106 to Therap Translation Services 105, which then forwards the data to the Internet 103 via an API/SFTP 104 server. Data from the Internet 103 first passes through a Router 110 and is then processed by two Firewalls, 115 and 116. Data going through Firewall 115 is directed to the Operations Servers 109 and the Database Server 114, which in turn connects to the primary Database Storage 108, Secondary Storage 113, and Tape Backups 112. In parallel, traffic passing through Firewall 116 is sent to a Load Balancer 111 that distributes requests to the Application Server Pool 118. From Firewall 115 the data goes back and forth to Router 117 and from there the data goes back and forth to Redundant Link to Hot Backup Site 119. Similarly from Firewall 116 the data goes back and forth to Router 117 and from there the data goes back and forth to Link to Hot Backup Site 119.
FIG. 1A illustrates the Setup for Face Recognition 122 system, which uses an Application Server 124 and a Front End Server 140 to manage automated attendance based on entry/exit sensors and facial recognition.

The following sections of the text describe this overall system and its operational flow.

Therap Attendance Demo System Overview: The ability to automate attendance logging was demonstrated at the 2025 National Conference in February 2025. Demonstrations were given during Automated Attendance Sessions for which conference participants signed up. Video sensors controlled by a machine-learning Front End Server (Face Recognition and Entry/Exit Detection) 140 that recognized attendee's faces in order to record attendance. This section describes the APIs between Therap Services Main App 123 and the Front End Server 140 to implement the demo.

A user working with Program Attendance expects to control all aspects of Attendance from Therap, which includes all steps for service authorization, Upload Photos 130 of individuals, and controlling when the attendance cameras are active. Therefore all UI aspects of the demo were with the Therap Services Main App 123.

The Front End Server 140 is preferably connected to a Therap Services Application Server 124. For a mobile application, Front End Server 140 functionality depicted in FIG. 1A is preferably integrated into the Therap Services Main App 123 running on one or more of the Therap Application Servers shown in FIG. 1.

Step 1: Program Setup: For the demo, the setup was enhanced to include a Front End sensor ID (i.e. a camera ID). Entrance Sensor 131 and Exit Sensor 132 were placed in rooms where programs were held. Associating a sensor with a program was done manually in the Therap back end. The Front End included a mapping of sensor ID to a room. Sensor ID format was a unique UUID. FIG. 1B details the specific, internal process that occurs on the mobile device during the Registration 141 step. The following text describes the process of capturing photos, converting them into machine-readable data, and validating them.

Step 2: In the Face Recording (Technical Process) the Therap Services Main App 123 has the ability to associate at least three face photos with an individual. Faces were converted to numeric vector embeddings via execution of a ML model on the image. This functionality was supplied by a Front End ML Embedding API 136. The endpoint accepted. Between 4 and 10 facial images, with minimum horizontal and vertical resolution of 500 pixels, and maximum of 3,000 pixels. The face preferably occupied at least 160 pixels. This process also includes IDF form ID and Provider Code as well. When ML embeddings did not already exist for the given IDF Form ID and provider code, they were added to a vector database. When embeddings already existed for the given IDF Form ID and provider code, they were replaced in the feature vector database with the newly generated embeddings. A use case for this is a person's appearance changes with age, they grow a beard, etc. The API returned to the status code with success/failure, and reason for failure (no face detected, more than one face detected, resolution not supported, etc.). Embeddings did not take a long amount of time to generate, so there was Embedding Generator 148 in the foreground.

Step 3: Enable/Disable a Sensor: A Therap Services user was able to enable or disable a sensor. Disabling a sensor ceases the video as well as all Front End events emitting from it. The endpoint of Front End Embedding API 136 enables or disables a sensor. Endpoint is accepted based on Sensor ID and enable or disable indicator. The API was idempotent, i.e. enabling a sensor that is already enabled has no effect and returns “Success.” Therap enabled another endpoint to check the status of a sensor. Endpoint accepted with Sensor ID: Endpoint returned whether sensor is enabled, disabled or sensor ID not found (404).

Step 4: Record presence of a person: The Front End had the following data setup. Each camera was associated with a provider code. Each provider code was configured with a single URL to invoke when a person's presence is detected. If this URL is empty, the Front End does not send any web hook. As the Front End recognizes people, it knows the provider code associated with the recognized person, and knows whether a Therap callback URL is configured. Therap Services provided an API to accept the presence of a person. The API endpoint accepted based on Provider Code, IDF form ID, Start Date/Time (UTC), Duration of recognition event (seconds), Sensor ID. Therap Services automates “checking the box” for an individual's attendance based on the above input.

Attributes includes if a person is not recognized, the Front End does not call the Therap API. If a person is not associated in the Front End with a provider with a webhook configured, the Front End Server 140 does not execute the webhook (calling Therap Services API). Timeliness includes the chance that face recognition with a lower confidence score may later change to a different person. Therefore it is preferred to defer reporting a person's attendance to Therap until the end of a “session” (duration of a person in the frame), to allow a potential correction to occur. Note this case is a rare occurrence.

Camera configuration has two approaches to monitoring attendance; one is to perpetually monitor presence in a room, which requires camera coverage of the entire room, without blind spots. The other approach is to monitor entry and exit only. The demo used the second approach. Based on testing, demo participants (attendees) were instructed to walk naturally past a camera without having to intentionally look or stare at the camera. However, the person was instructed to face as close to square with the camera as possible. A slight angle was acceptable. The images taken to create embeddings were taken with the person facing the camera. The Therap Attendance demo had the ability to record time-in and time-out. This was done with two cameras, one camera faces people entering for time-in, and the other faces people exiting—time-out. Of course, this did not take into account situations, for example, if someone leaves to use the bathroom or get coffee, or if there are two sessions on the same day. For this demo, every entry and exit was logged.

API Security: Therap Services leveraged its existing API Token Mechanism. A signed JSON Web Token (“JWT”) was associated with each provider code. The Front End must “login” with the JWT before invoking the webhook. The JWT was created manually and was given to the Front End team manually. The Front End provided API security.

The Therap Video Attendance Demo User Experience is described below.

Attendance Set Up 125 (Behind the Scenes): Programs were created in advance in Therap Setup, and a pool of individuals assigned to each program. Each Program was a session. The site was a conference room. Therap Conference attendees were pre-populated into the demo provider as “individuals.” A Therap Program had one or more sensor IDs associated with it. Therap Services knew which sensors were for “Entrance Sensor 131” and which were for “Exit Sensor 132”. At the conference, there were multiple sessions per room per day. Worst case, one session per day was associated with a program. Best case, the start/end time of a conference session was associated with a specific program.

The Front End application associated each sensor with a Therap “Check-in/Check-out” API Endpoint in the ML setup, which was a Front End Server 140 to Therap Attendance.

Face Recording: Conference Attendees who attended the Therap Automated Attendance Session were asked to volunteer to participate in a Therap Video Attendance Demo. Before the demo began, participants were directed to a kiosk to have their picture taken. At least three uploaded photos 130 were added on Therap Individual Profile using a Therap Services Main App 123. The participants were asked their names, and the photos were associated with the “Individual” pre-loaded into a Therap demo provider with each participant's name. The photos were then uploaded to the Front End to create facial embeddings. An IDF form ID and provider code were associated with the Front End vector embedding data of the person's face. Each participant was instructed on how and when to present themselves to the sensor.

Meeting Room Setup: Two sensors were used in the Automated Attendance Session, an “Entrance” and “Exit.” They were clearly labeled as “Entrance Sensor” 131 or “Exit Sensor 132”. The sensors were placed on tripods in the front of the meeting room, providing a good view of the demo for all attendees. The Entrance Sensor 131 faced one direction, emulating capturing of room entry. The Exit Sensor 132 faced the opposite direction, emulating room exit. Cordons may be used to direct people to ensure their faces are in full view of the sensors upon entry/exit.

Entry/Exit Recognition: During entry time (preferably, 10 minutes before through 10 minutes after the start of a session), the Entrance sensor 131 is enabled, and Exit sensor 132 is disabled. For the demonstration portion of the session, each person was instructed to walk naturally past the sensor, and to continue until out of view of the sensor. Leaving the sensor's field of vision is important, since that triggers the sensor to send data about the recognized person event to Therap. The Front End Event Processor App 135 received a recognized person event containing the sensor ID and ML embedding. The Front End Event Processor App 135 sent the embedding to the Front End Embedding API 136 for recognition. The Front End Embedding API 136 performed a nearest neighbor search to find a matching embedding in the feature vector database with a probability of a match. The Front End application sends a recognized person event, date/time (UTC) and sensor ID after the recognized person leaves the video frame. Therap Services translates UTC time to Program Time Zone. Therap Services maps the sensor ID to Entry/Exit. An attendance dashboard is integrated with the system, where a screen in the room shows attendance status. Once all volunteers simulated entering the room by walking past the “Entrance” sensor 131, the Entrance sensor 131 was disabled and the Exit sensor 132 was enabled. Participants were then asked to walk past the Entrance sensor 131 until exiting from its field of view. When a session wraps up, Therap Attendance is viewed to show entry times. Preferably, the Therap Services Main App 123 handles duplicate events. For example, if the same person enters the room three times as a session starts (getting coffee, etc.), the Therap Services application uses the first time as the Attendance start time. Similarly, the last Exit time is used as the Attendance end time.

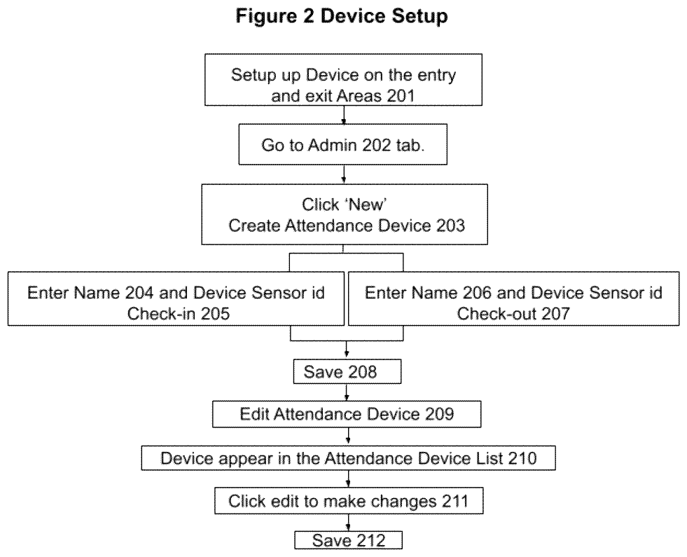

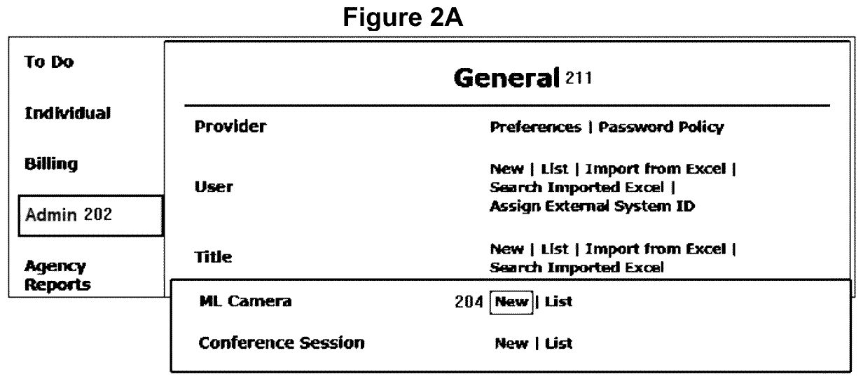

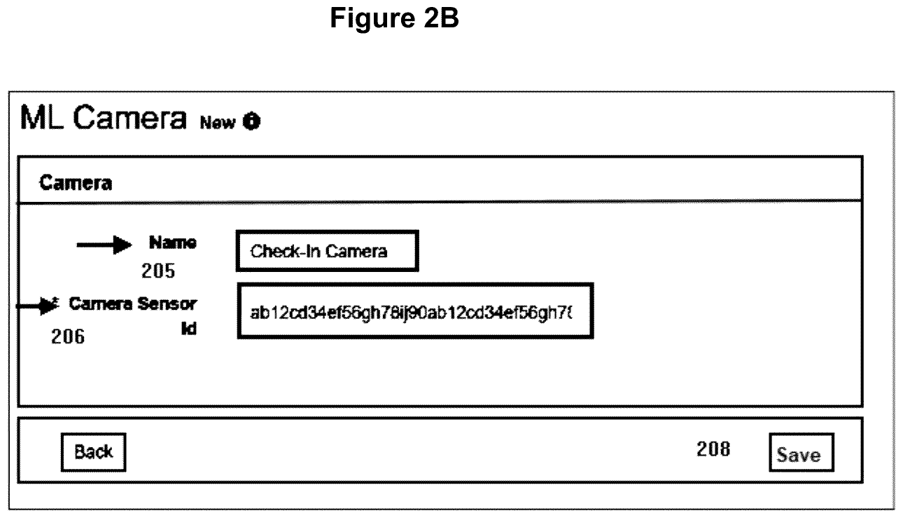

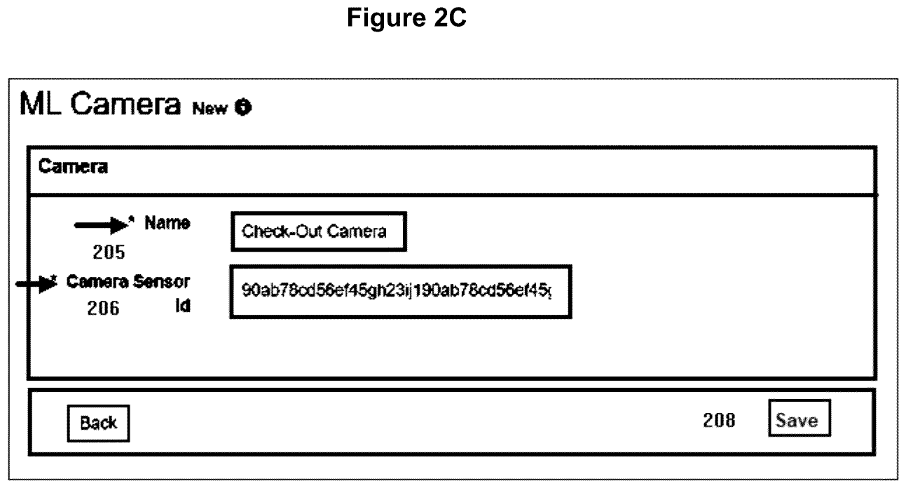

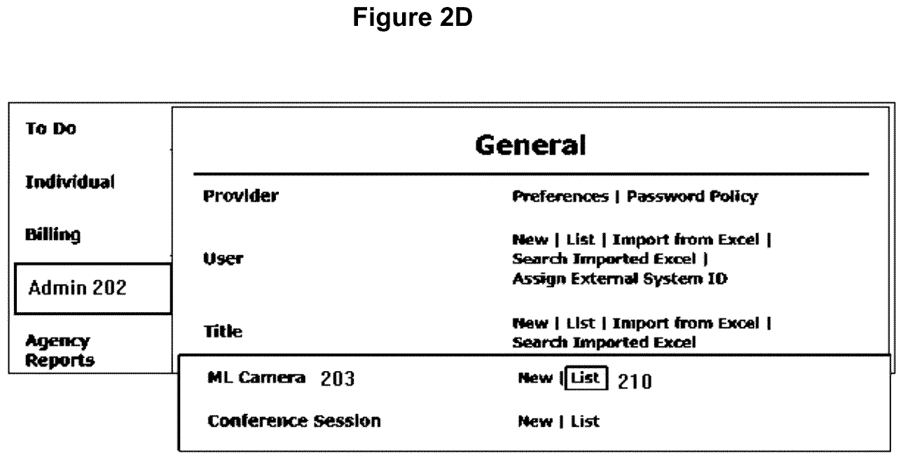

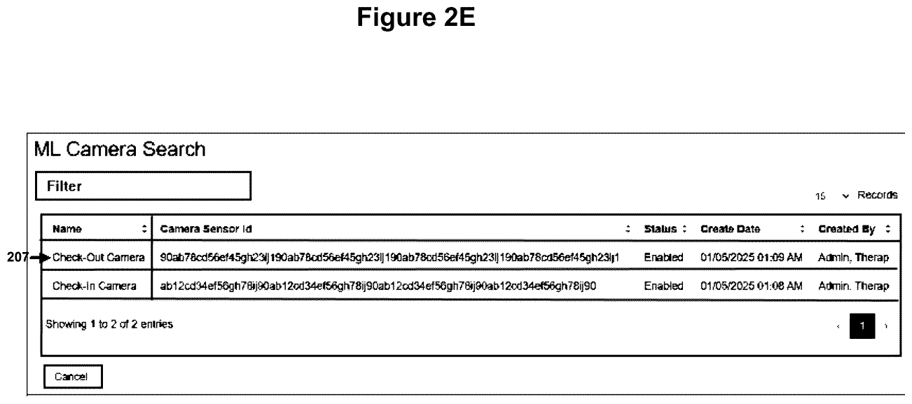

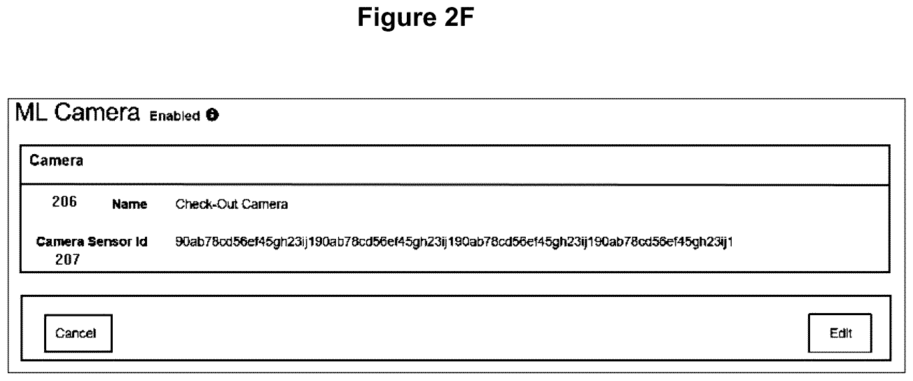

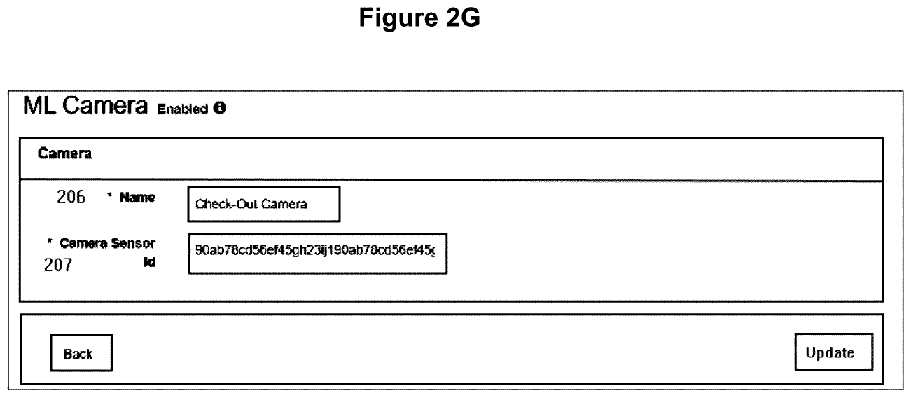

FIG. 2 illustrates how the system is set up to use devices that automatically record attendance using facial recognition. In this example, the devices are being prepared for a conference session, where attendees check in at the start and check out at the end. The same process may also be applied to other activities such as classes, service programs, billing-related visit tracking, or verifying staff and participant presence for compliance purposes. Two devices are linked to the session—one for check-in and one for check-out. These devices may be fixed cameras installed at the entry and exit points or mobile devices such as smartphones running the Therap Attendance application. Fixed cameras are positioned so they clearly capture individuals entering or leaving, while mobile devices may be placed at a temporary checkpoint or moved between locations as needed. Before recording begins, each device is connected to the internet and registered in the system. The connection is secured through an API that uses token-based authentication, and in some cases, the device may process the image locally and send only an encrypted or hashed version to the server.

Set Up Device on Entry and Exit Areas 201: As shown in FIG. 2A 202, the first step is to prepare the check-in and check-out devices so they are ready to capture attendance events. For fixed devices, this includes mounting the camera, aiming it at the correct location, ensuring adequate lighting, and confirming it is powered and online. For mobile devices, this includes installing the Therap app, connecting the device to the network, and pairing it with the system using its unique ID. In both cases, the administrator assigns a Device Name 204 and a Device Sensor ID Check-in 205 for the entry device, and a Device Name 206 and Device Sensor ID Check-out 207 for the exit device.

Create Attendance Device 203: In FIG. 2B 205, the administrator initiates the process to create a new attendance device. FIG. 2B shows the form where the Enter Name 205 and Device Sensor ID Check-in 206 are entered for the check-in device. FIG. 2C shows the form where the Enter Name 205 and Device Sensor ID Check-out 206 are entered for the check-out device. After entering the required details, the administrator clicks Save 208 to register the devices in the system. For mobile devices, pairing may involve scanning a QR code or entering a token to confirm authorization. Depending on the setup, devices may be configured to send either full images or hashed facial data to the server.

Edit Attendance Device 209: FIG. 2D shows the Attendance Device List 210 in the Admin 202 interface. From here, the administrator may select a device 207 to view or modify its details. FIG. 2F shows the existing configuration form, and FIG. 2G shows the updated form after edits have been made and Saved 212. Edits may include changing the device name, reassigning it to a different session or site, switching between fixed and mobile operation modes, or updating its operational status. Once saved, the changes take immediate effect.

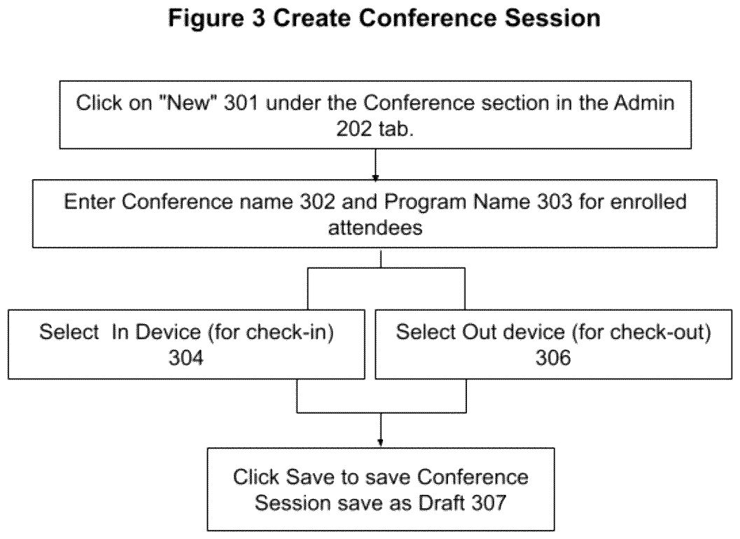

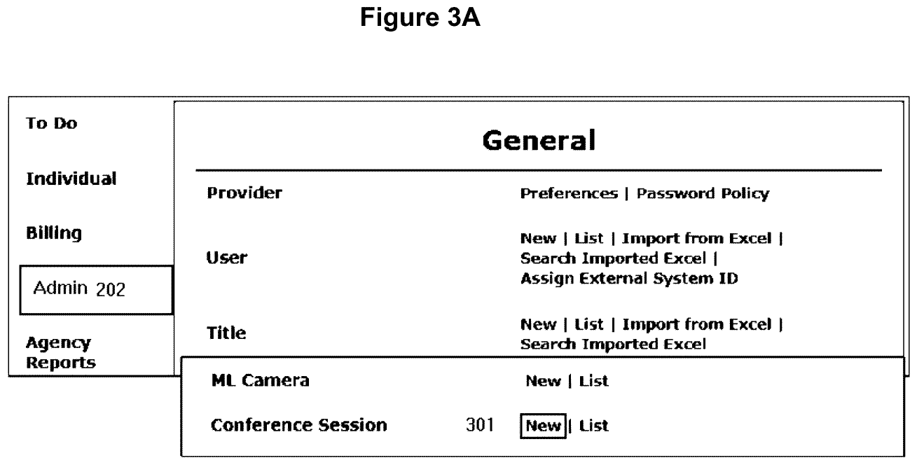

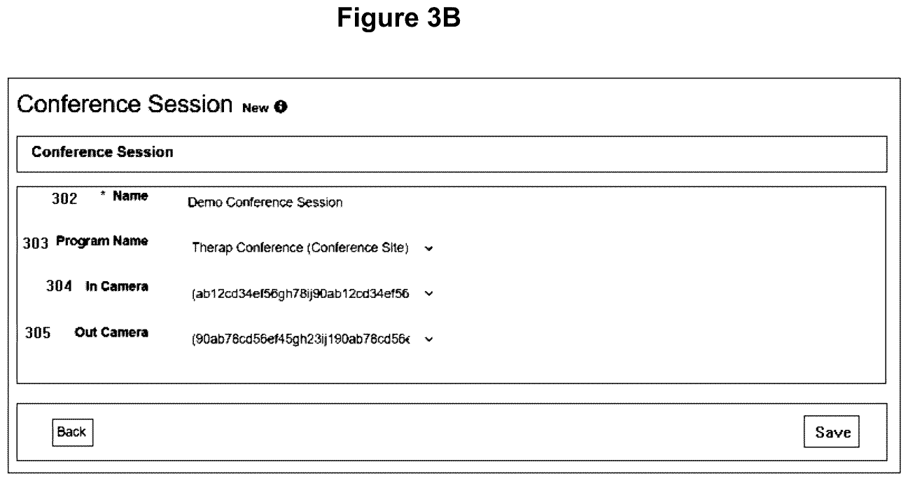

FIG. 3 shows how a session is created and linked to the devices for automated attendance capture. While this example focuses on a conference session, the same process may be used for other activities such as training sessions, service programs, or any event where attendance needs to be tracked automatically.

Create Conference Session: As shown in FIG. 3A, the administrator clicks on New 301 under the Conference Session 302 section in the Admin 202 tab.

In FIG. 3B, the administrator enters the Conference Name 302 and the Program Name 303 for the enrolled attendees. The configured In Camera 304 device is selected for check-in, and the configured Out Camera 305 device is selected for check-out. Devices may be fixed or mobile depending on the setup. Once all details are entered, the administrator clicks Save 306 to save the conference session in draft mode.

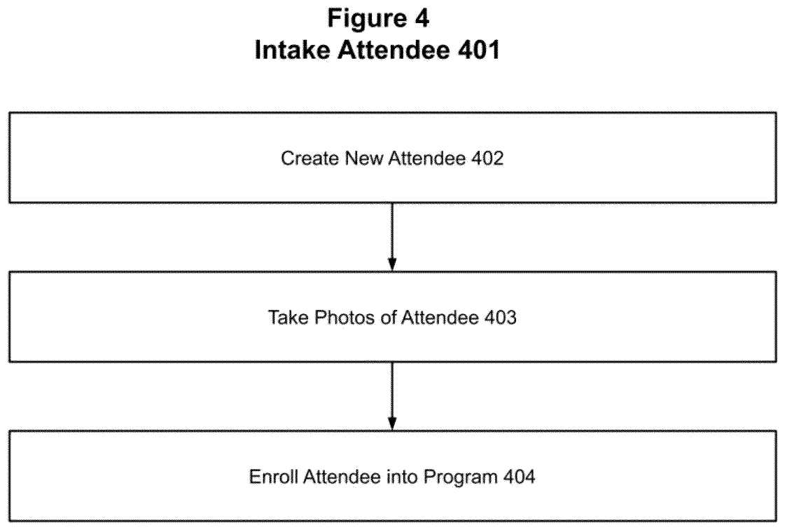

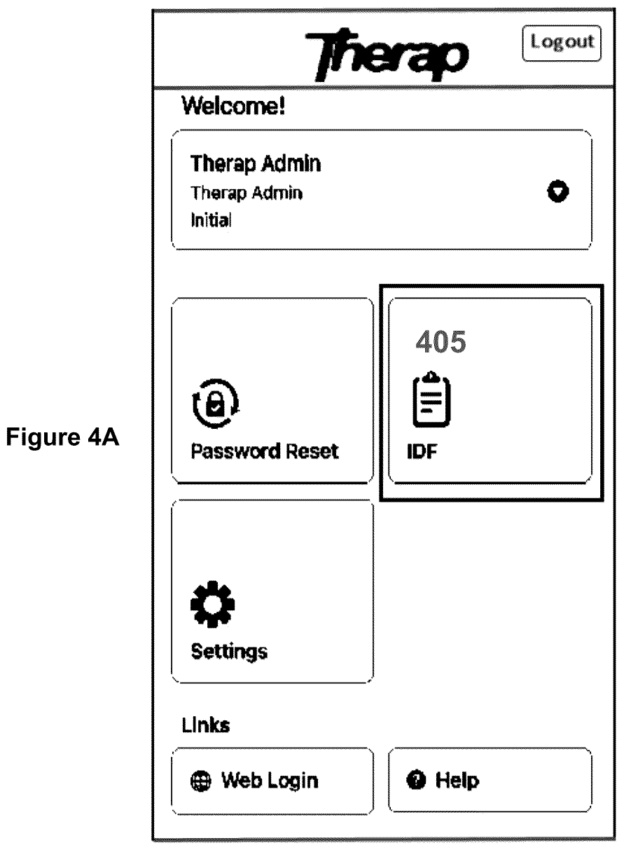

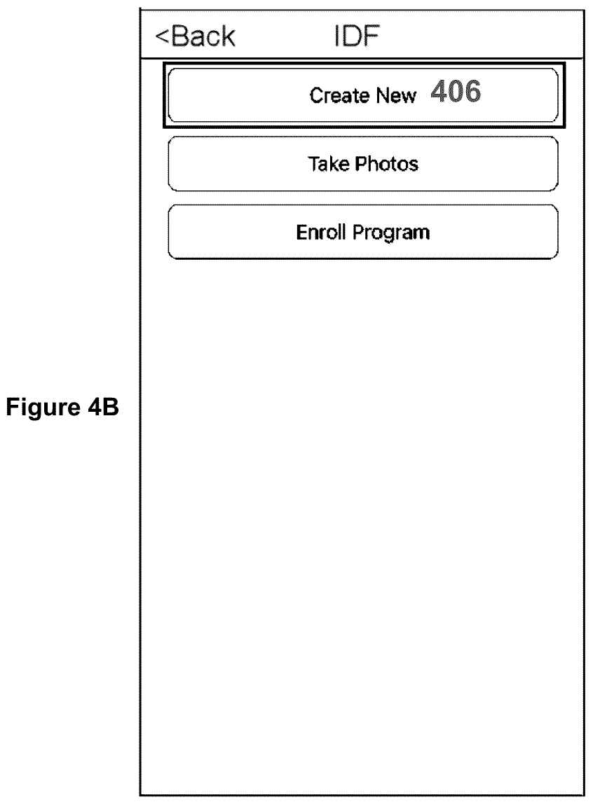

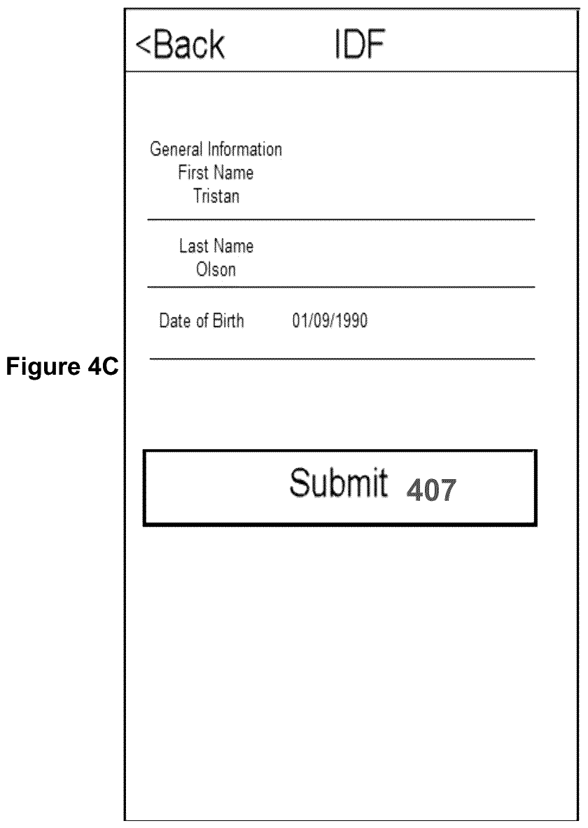

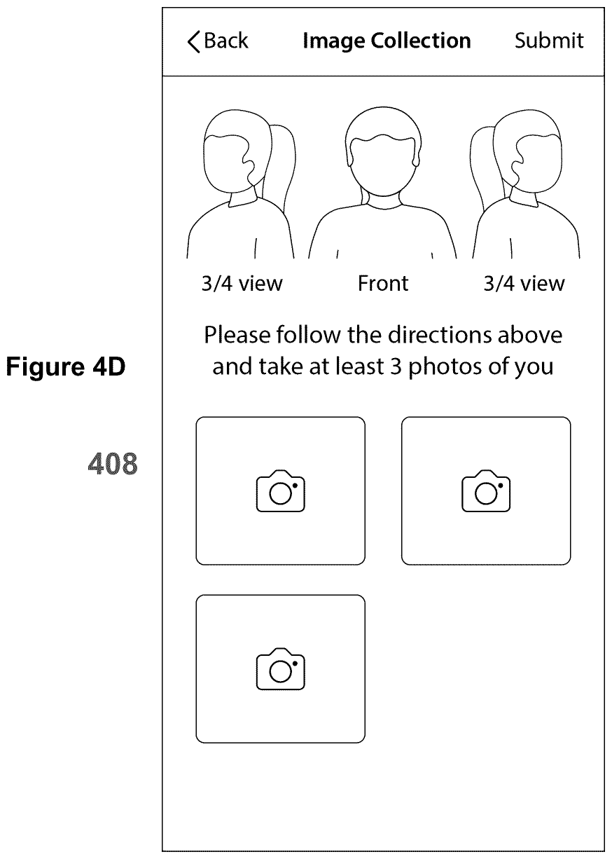

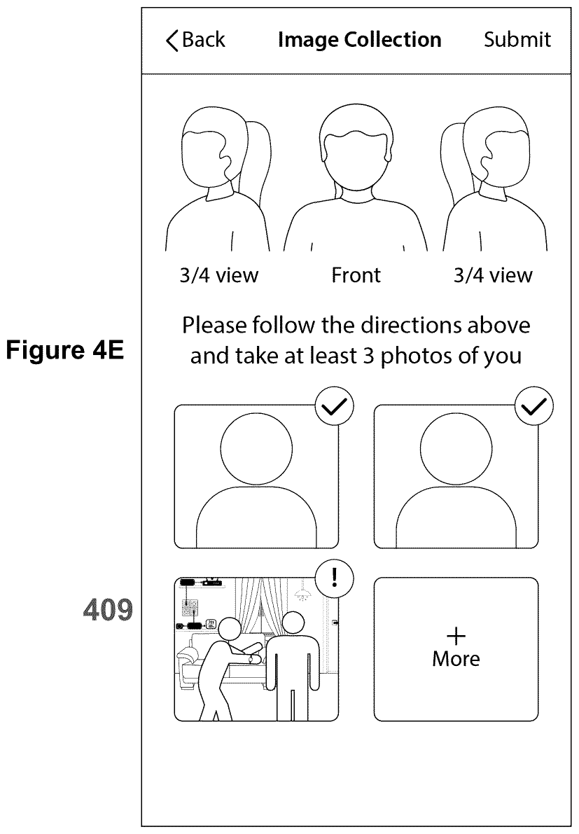

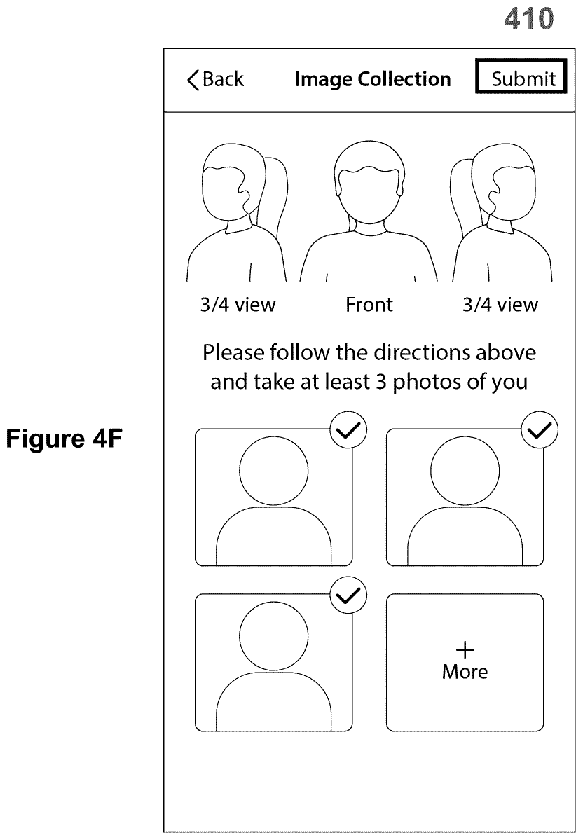

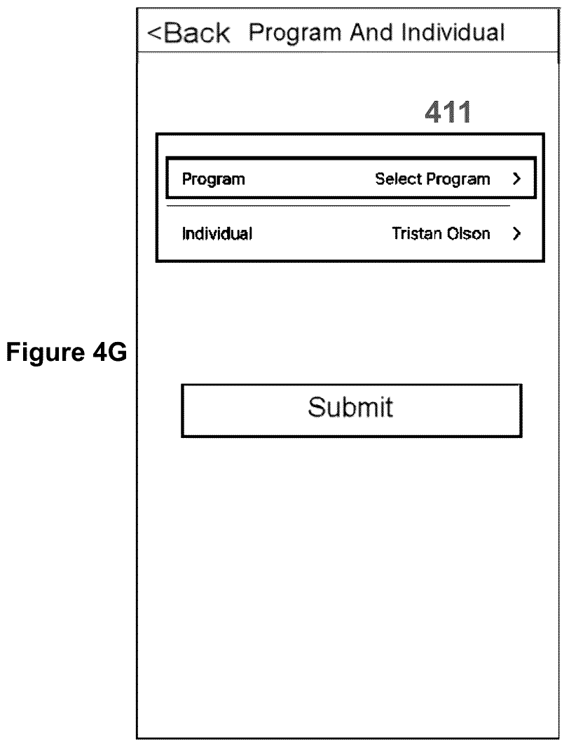

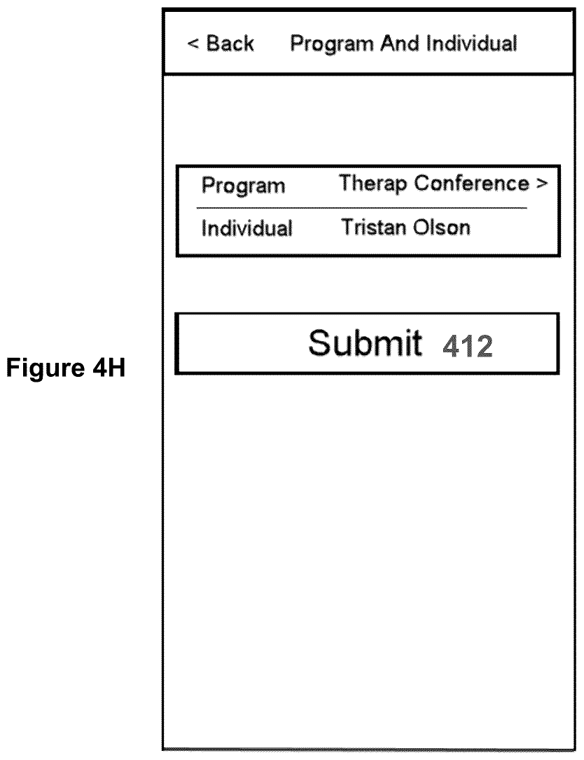

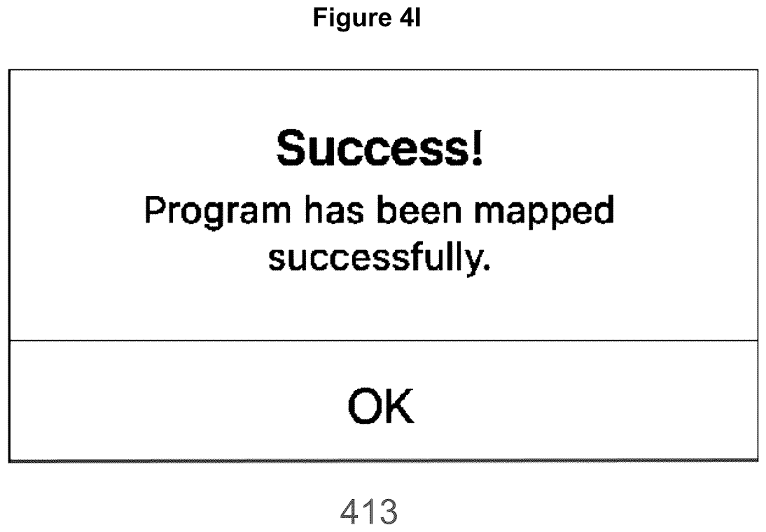

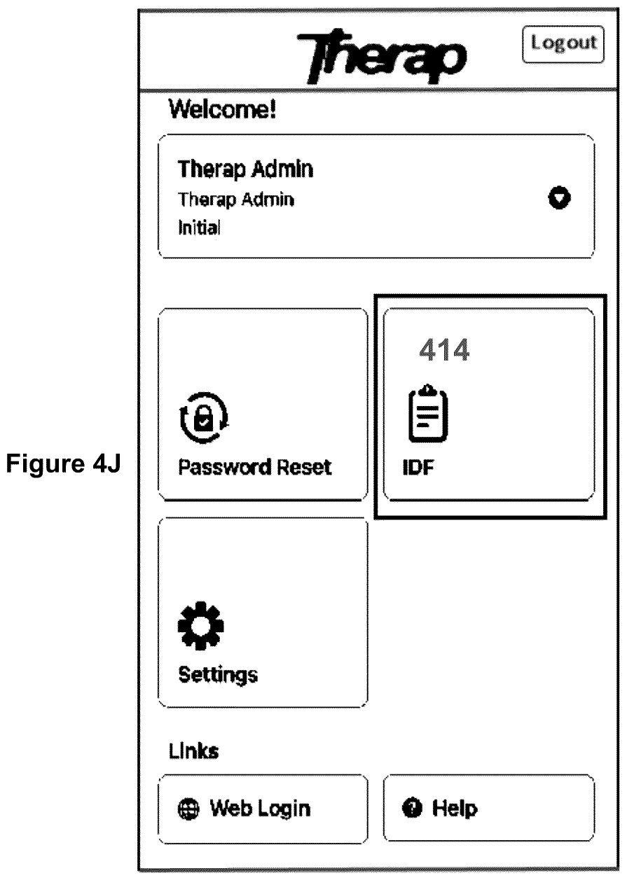

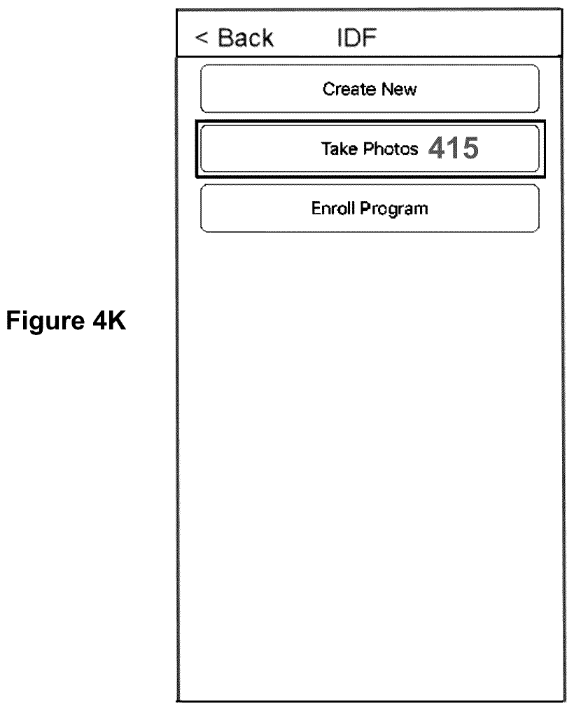

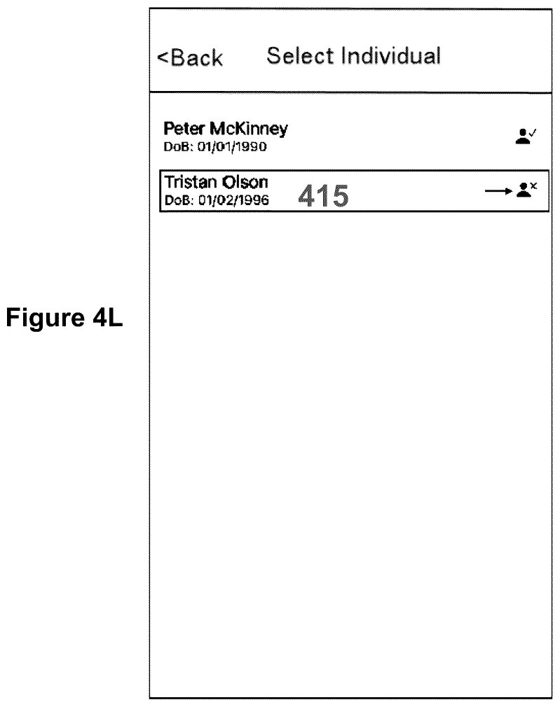

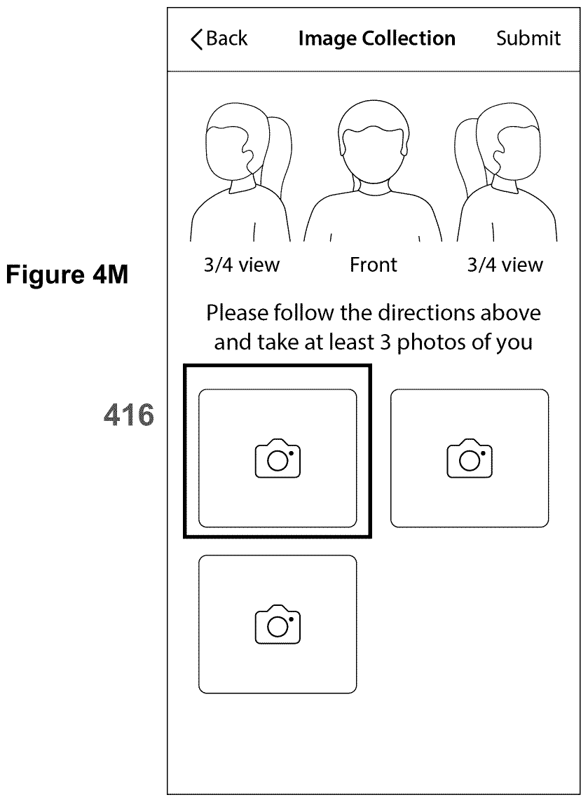

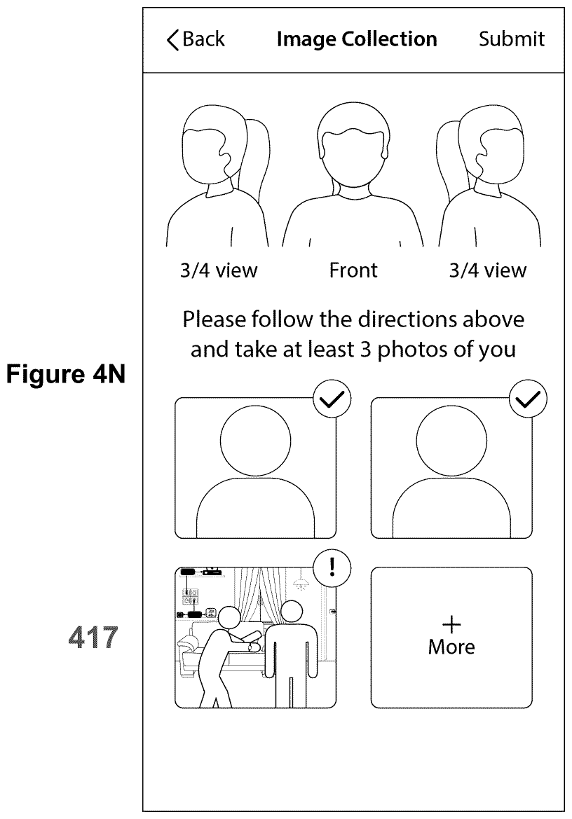

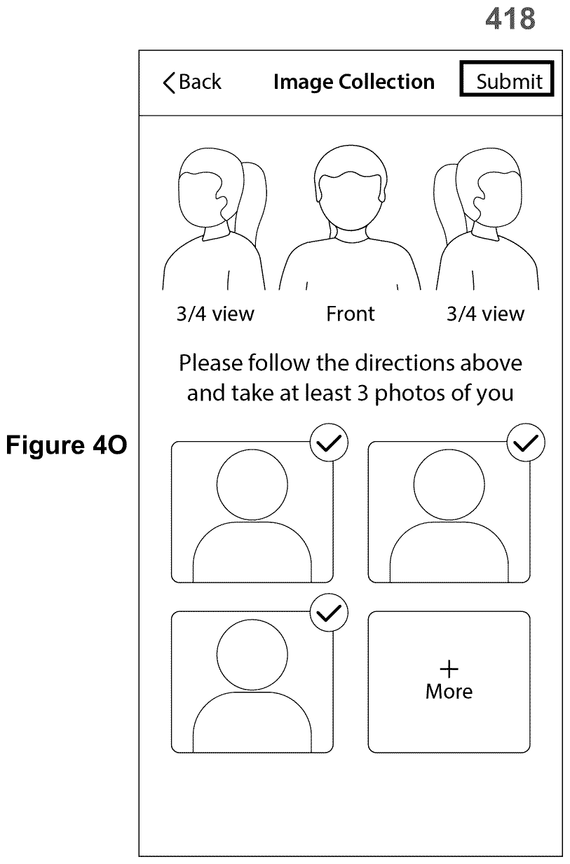

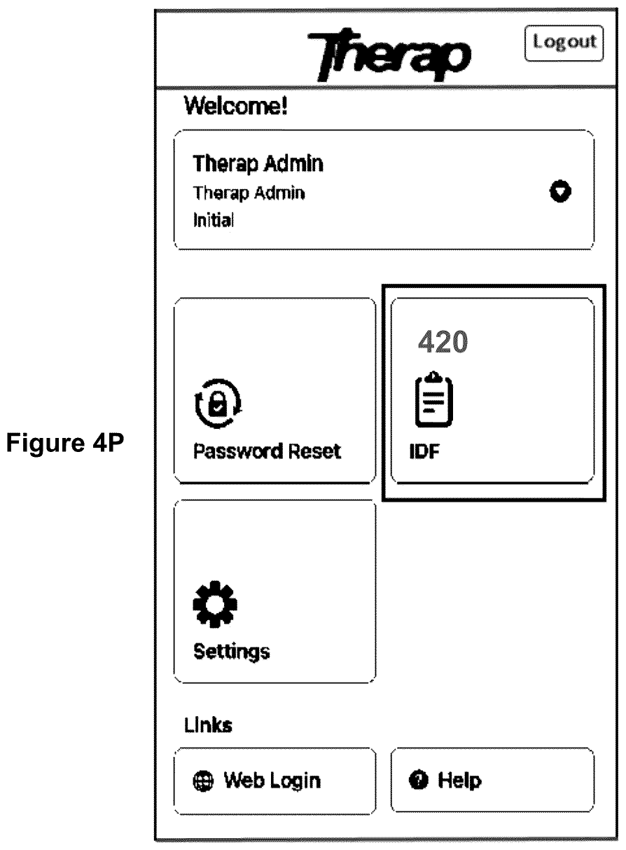

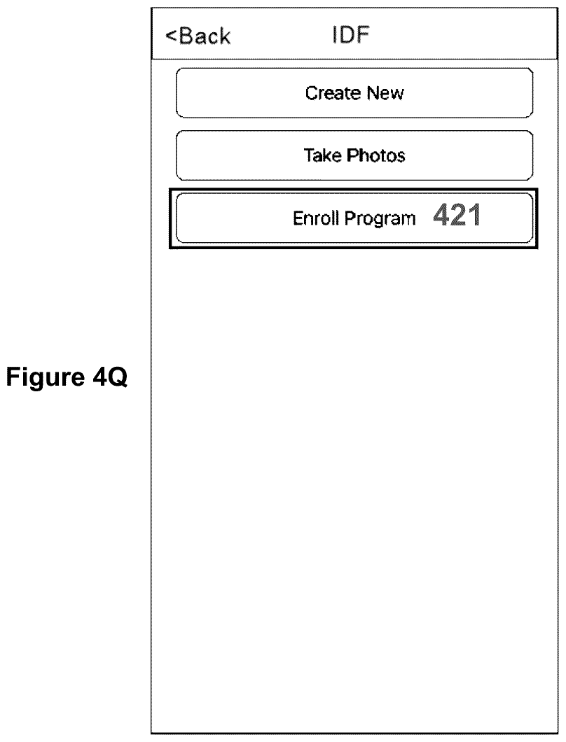

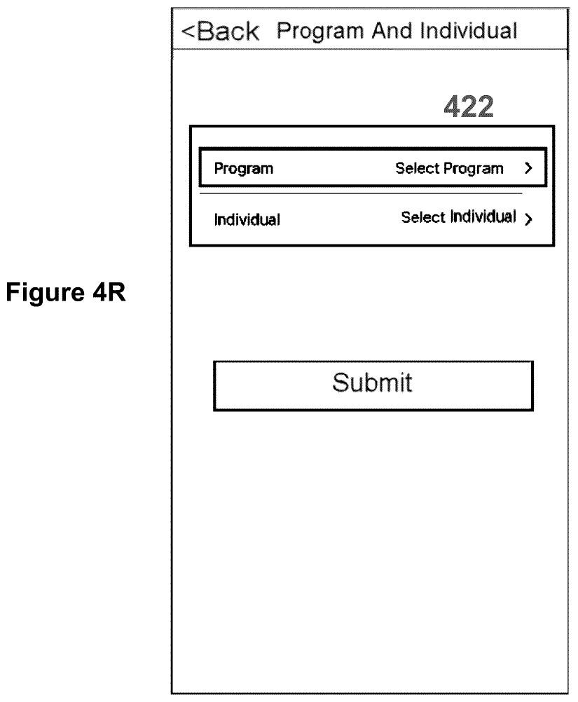

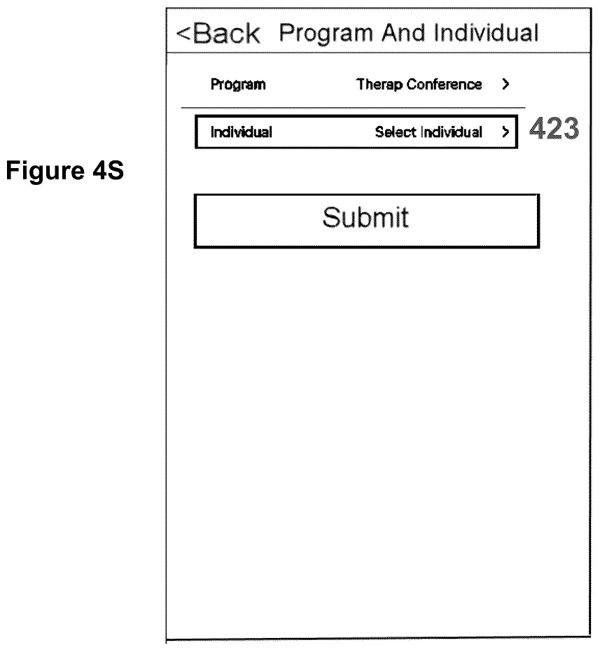

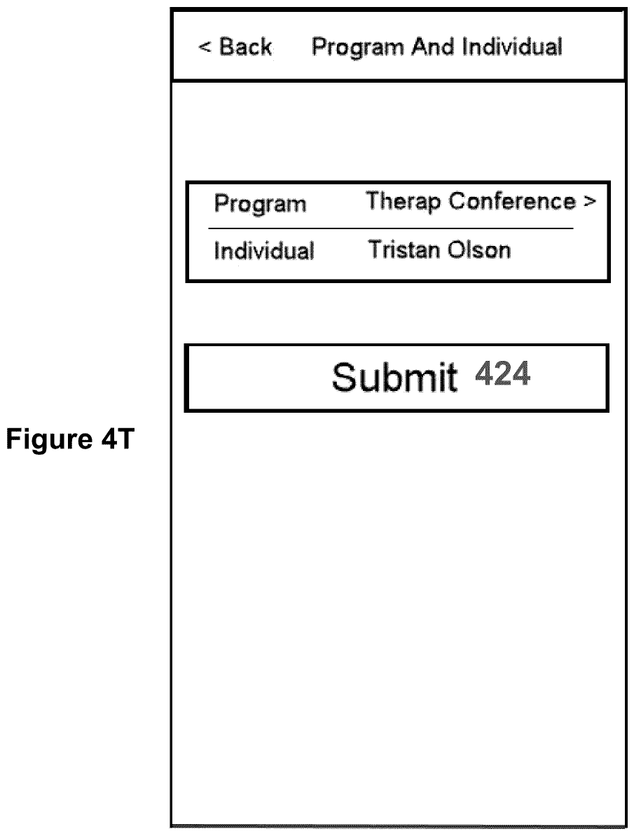

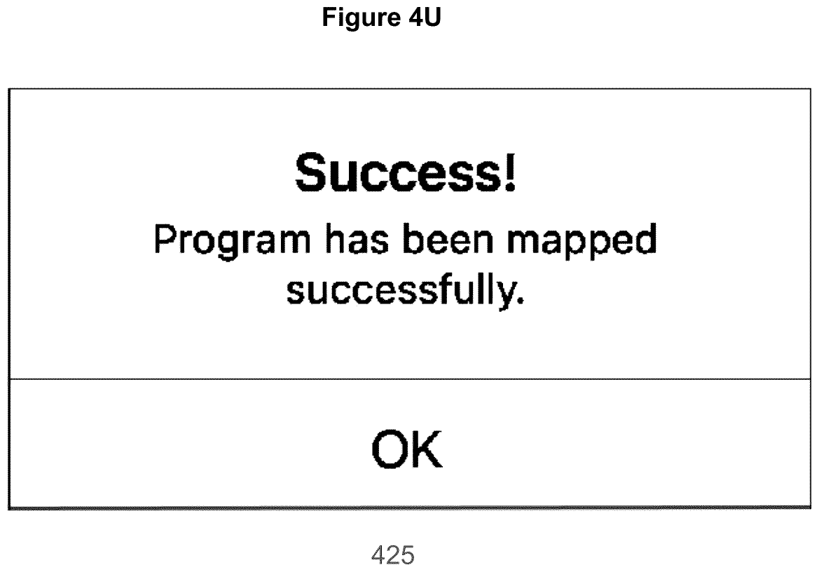

FIG. 4 Intake: Attendee 401 depicts how to intake attendees. A user needs to Create New Attendee 402 until it is ready to be activated. Next, a user needs to Take Photos of Attendee 403. Lastly, the user needs to Enroll Attendee into Program 404 in order to complete the intake procedure.

Attendees are to be entered into the system along with their photos and enrolled into the specific Program for the conference, preferably using the Therap iOS Mobile Application, before they can check in to a Conference Session.

Create New Attendee: As shown in FIG. 4A, the process begins from the mobile app dashboard by navigating to the IDF 405 tab.

In FIG. 4B, the following screen provides the Create New 406 button, which leads to the attendee entry form.

As illustrated in FIG. 4C, the user is presented with a form to enter the attendee's First Name, Last Name, and Date of Birth, after which the form is submitted by hitting the Submit 407 button.

Following this, as shown in FIG. 4D, the interface displays camera 408 icons which are used to begin capturing photos of the attendee's face. A minimum of three photos are required.

FIG. 4E depicts how previously captured photos may be retaken by selecting them again, if necessary. It is important to ensure that no other faces are visible in the photos. If multiple faces are detected, an error icon appears, indicating that the photo must be retaken 409.

In FIG. 4F, once the photos are taken, the form is submitted by hitting the Submit 410 button, as shown in the same figure.

In FIG. 4G, the Program 411 field is used to select the Program for the Conference Session in which the attendee is to be enrolled.

In FIG. 4H the attendee enrollment is confirmed upon submission when clicking on the Submit 412 button.

In FIG. 4I, a message appears saying “Success! Program has been mapped successfully” 413 indicating successful mapping to the Program.

Take Photos of Attendee: After entering an attendee's information, clicking on the Back button and then the Leave button on the confirmation pop-up only saves the entered information without any photos. Their photos may be taken later by following these steps.

As shown in FIG. 4J, the user needs to click on the IDF 414 tab on the mobile app dashboard.

In FIG. 4K, the user needs to click on the Take Photos 415 button in order to take pictures.

FIG. 4L shows the process of selecting the attendee whose photos have not been taken. Attendees who do not have photos in the system yet are marked with a cross icon on their right 416.

In FIG. 4M, the user needs to click on the Camera icons 417 on the next page to start taking photos of the attendee's face. A minimum of 3 photos should be taken.

FIG. 4N shows how to click on already taken photos to retake them if necessary. There should not be faces other than the attendee's in the photos. If a photo is taken with multiple icons, then there is an error icon on top of the photo 417 and that photo is to be retaken.

As shown in FIG. 4O the user needs to click on the Submit 419 button after the photos have been taken.

Enroll Attendee into Program: After entering an attendee's information and photos, the user needs to click on the Back button, and then the Leave button on the confirmation pop-up only saves the entered information and photos without enrolling the attendee to the required Program. They may be enrolled later by following the steps below.

In FIG. 4P, the user needs to click on the IDF 420 tab on the mobile app dashboard.

FIG. 4Q shows the interface having the Enroll Program 421 button.

FIG. 4R shows the Program 422 field and selecting the program for the Conference Session in which the attendee is to be enrolled.

FIG. 4S shows the user needs to click on the Individual 423 field and select the attendee to enroll.

In FIG. 4T, the user needs to click on the Submit 424 button to enroll the attendee into the respective Program.

A message “Success! Program has been mapped successfully” 425 is shown in FIG. 4U confirming that the attendee has been successfully mapped to the Program.

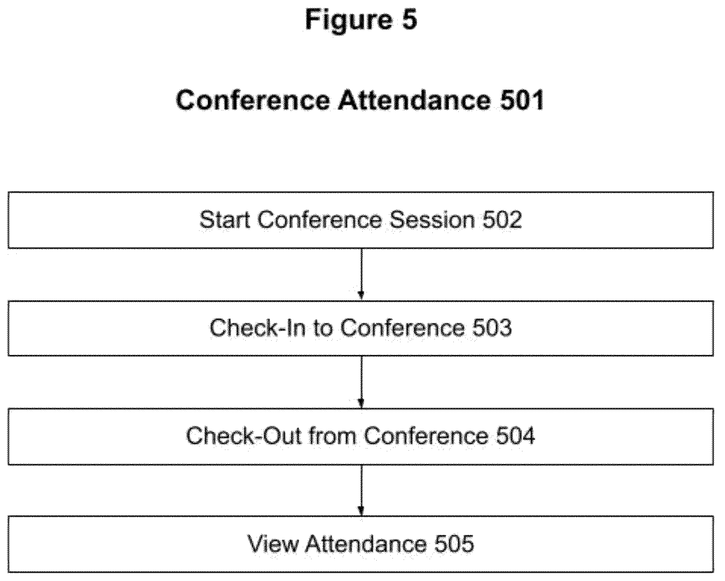

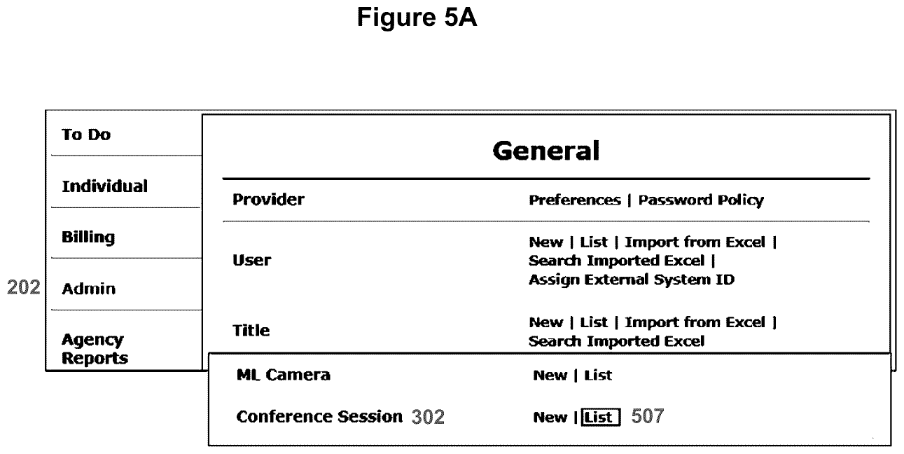

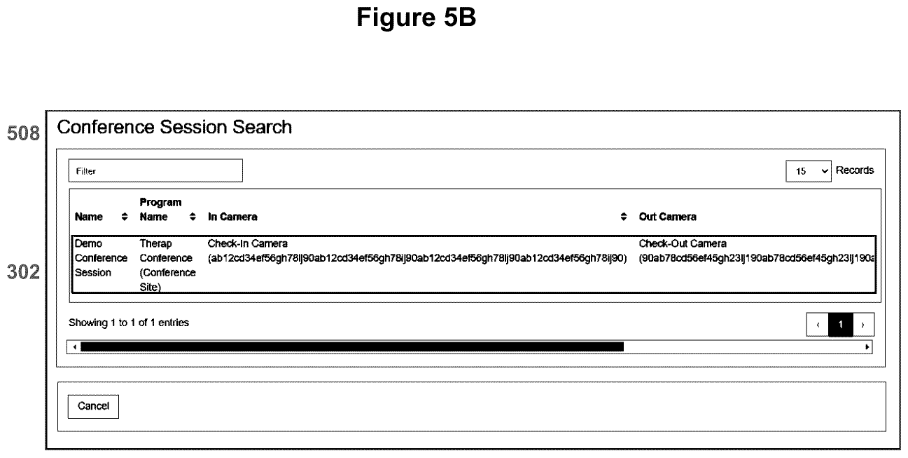

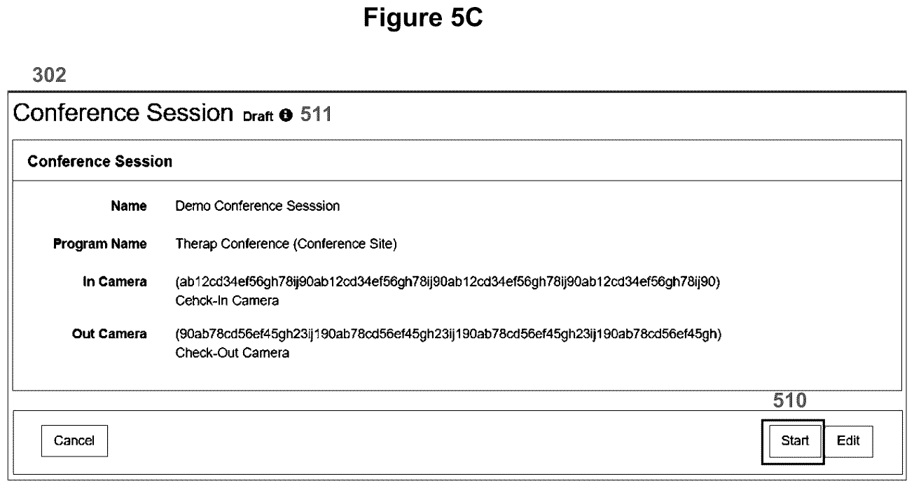

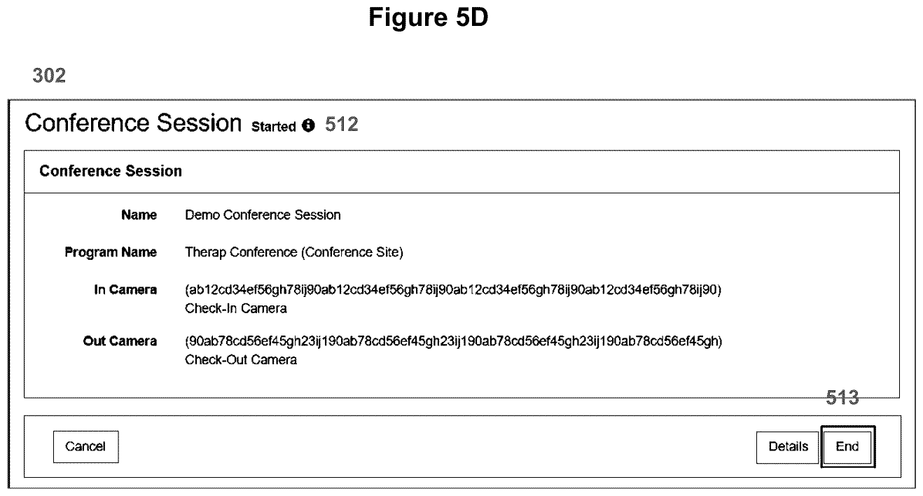

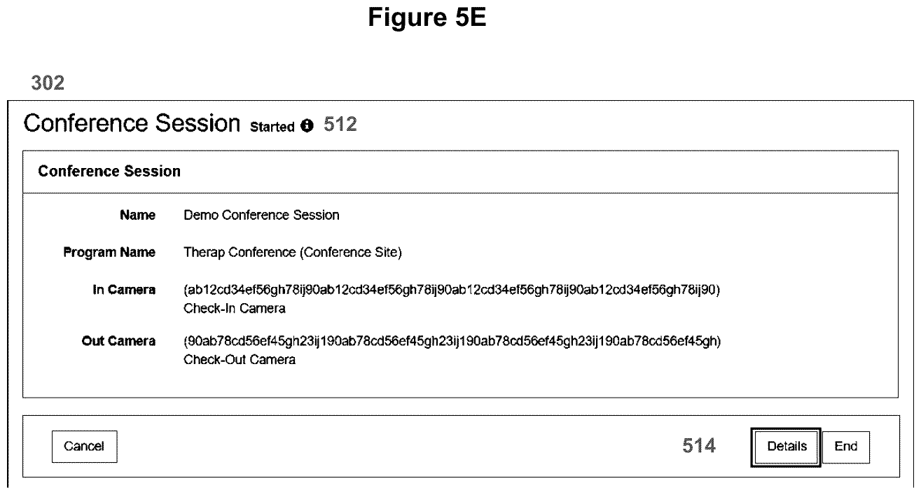

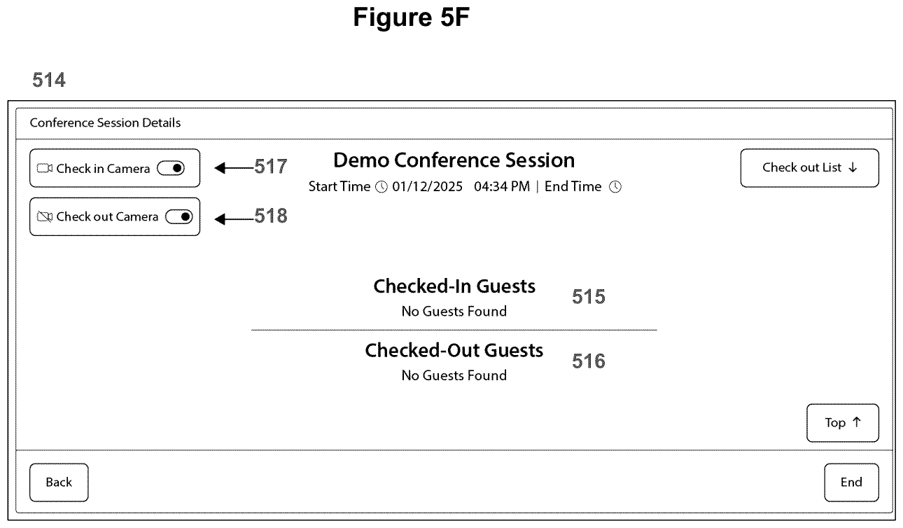

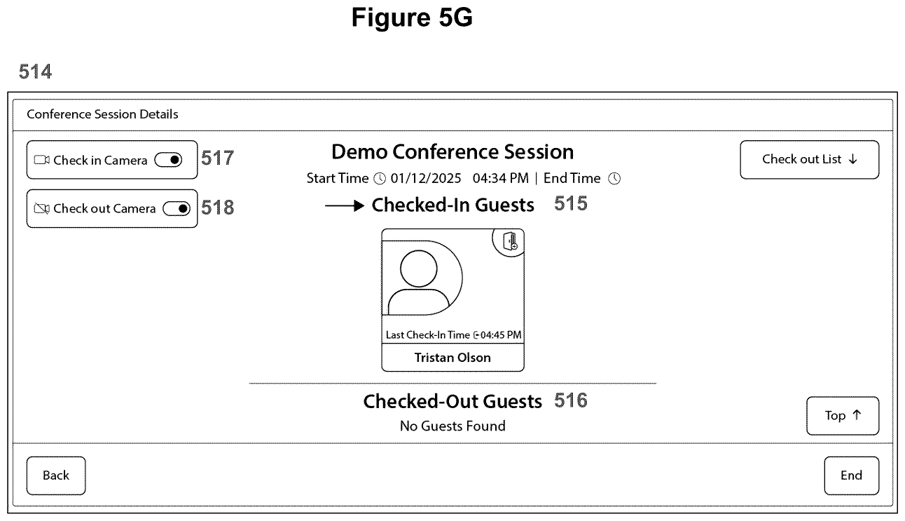

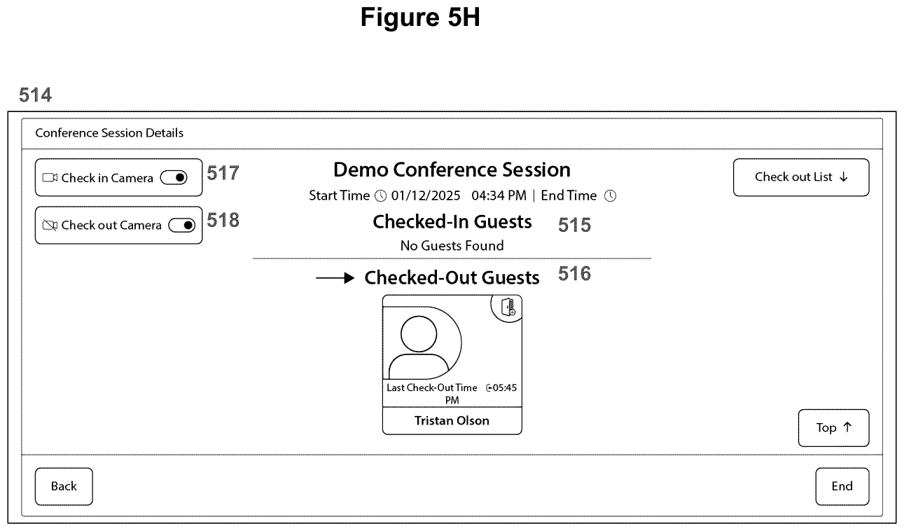

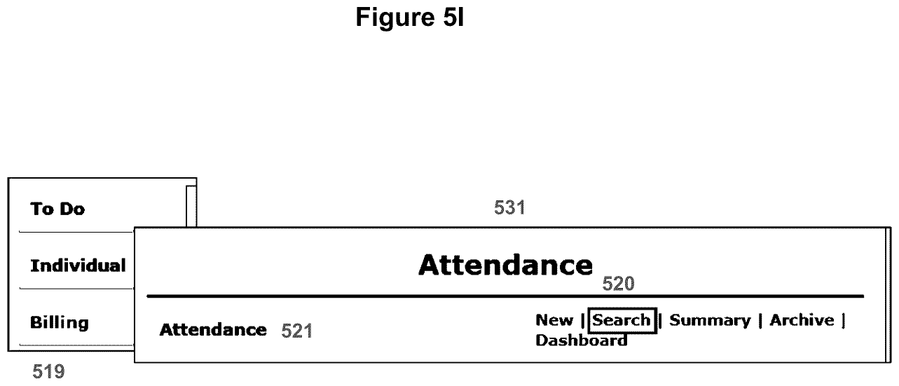

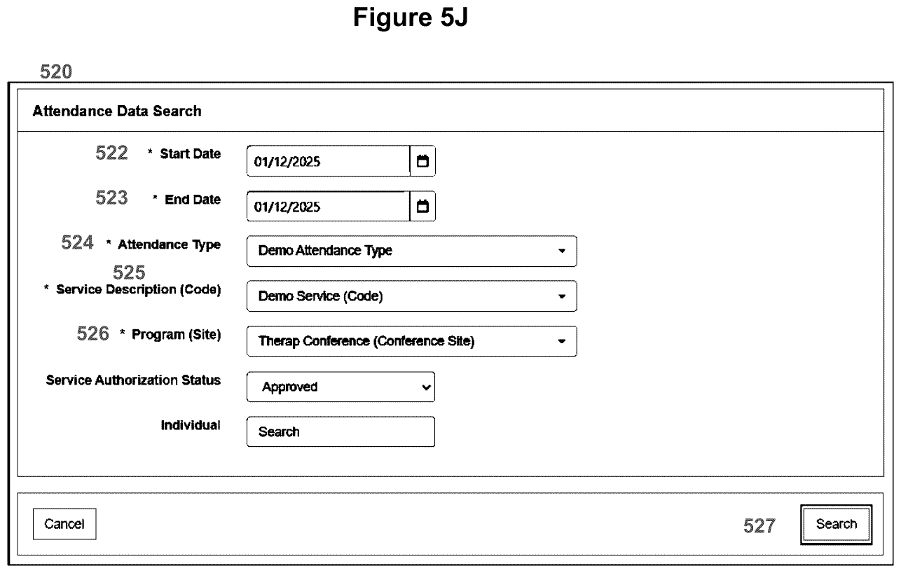

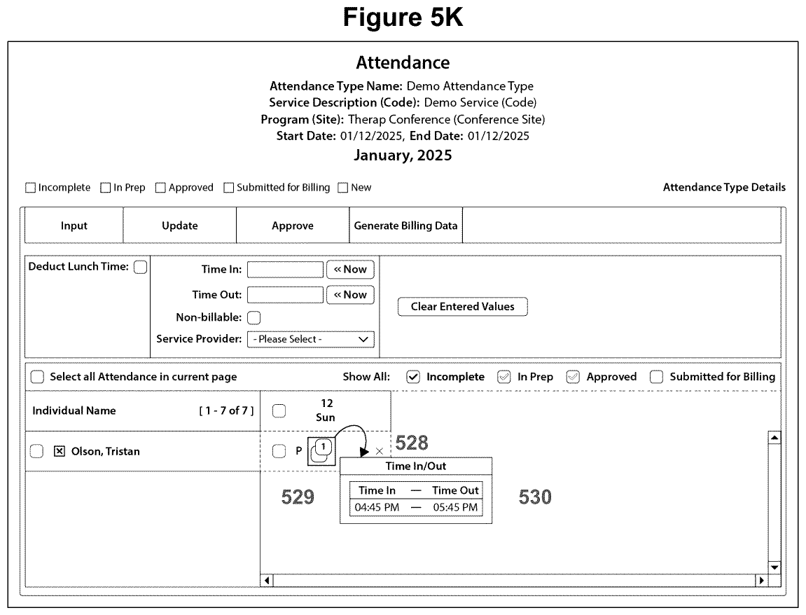

FIG. 5 illustrates the flow of Conference Attendance 501. As shown in the figure, the process of capturing attendance begins with Start Conference Session 502 to record attendance and Check-In to Conference 503 moving towards Check-Out from Conference 504 and concluding with View Attendance 505 to preview recorded attendance.

After the cameras have been set up and the attendees have been enrolled, the conference session 302 may be started to start recording their attendance.

Start Conference Session 502: In FIG. 5A, the process of Start Conference Session 502 begins from the Admin 202 tab. Users would need to click on the List 507 link by navigating to the Conference Session 302 option in the General 211 section.

FIG. 5B depicts the Conference Session Search 508 list would appear and users would have to click on the desired session from the Draft 511 Conference Session 302 listed.

In FIG. 5C, the Draft 511 Conference Session 302 form may be seen. Users may start recording Attendance for the session by clicking on the Start 510 button at the bottom right corner of the form.

FIG. 5D illustrates that multiple Conference Sessions 302 cannot be started simultaneously. In the event of an on-going session, its termination is required; this can be achieved by clicking on the End 513 button on the respective Conference Session 302 form.

FIG. 5E illustrates that once a Conference Session 302 begins, the form status would change to Started 512. Users are able to track the check-in and check-out by clicking on the Details 514 button.

As illustrated in FIG. 5F, users are able to see if attendees have checked-in and checked-out on the Conference Session Details 514 page. By clicking on the Check in Camera 517 and Check out Camera 518 toggles, users may turn on/off the check-in 517 and check-out cameras 518 at will. If a camera is turned off and turned on again, it starts recording after 1 minute of being turned on.

Check-In to Conference 503: FIG. 5G illustrates that once the Conference Session 302 begins, the Check in Camera 517 would automatically record the attendees as they walk in before the camera. It needs to be ensured that the Check in Camera 517 remains turned on and the attendees appear before the camera for at least 3 seconds for it to be able to successfully record them checking-in to the Conference Session 302. Photos of the attendees who have been checked-in to the conference would be shown in the Conference Session Details 514 page under the Checked-In Guests 515 section.

Check-Out from Conference 504: FIG. 5H illustrates that after checking-in, attendees checking-out from the conference would have to appear before the check-out camera 518 for at least 3 seconds for their check-out to be successfully recorded. It should be ensured that the check-out camera 518 remains turned on during check-out. Attendees who have successfully checked-in and then checked-out from the conference are removed from the Checked-In Guests 515 section and shown under the Checked-Out Guests 516 section.

View Attendance 505: The recorded attendance of the Conference Session may be viewed by the following steps: In FIG. 5I, the process of viewing Attendance begins by clicking on the Search 520 button beside the Attendance 521 option in the Attendance 531 section of the Billing tab. As displayed in FIG. 5J, on the Attendance Data Search 520 page, the date of the Conference Session 302 in the Start Date 522 and End Date 523 fields are entered along with the respective Attendance Type 524, Service Description (Code) 525 and Program (Site) 526, and clicked on the Search 527 button. FIG. 5K illustrated that the attendees, whose check-in and check-out has been completed, are shown on the Attendance grid. By hovering over the Time In/Out 528 icon, users may view the Time In 529 and Time Out 530 of the users.

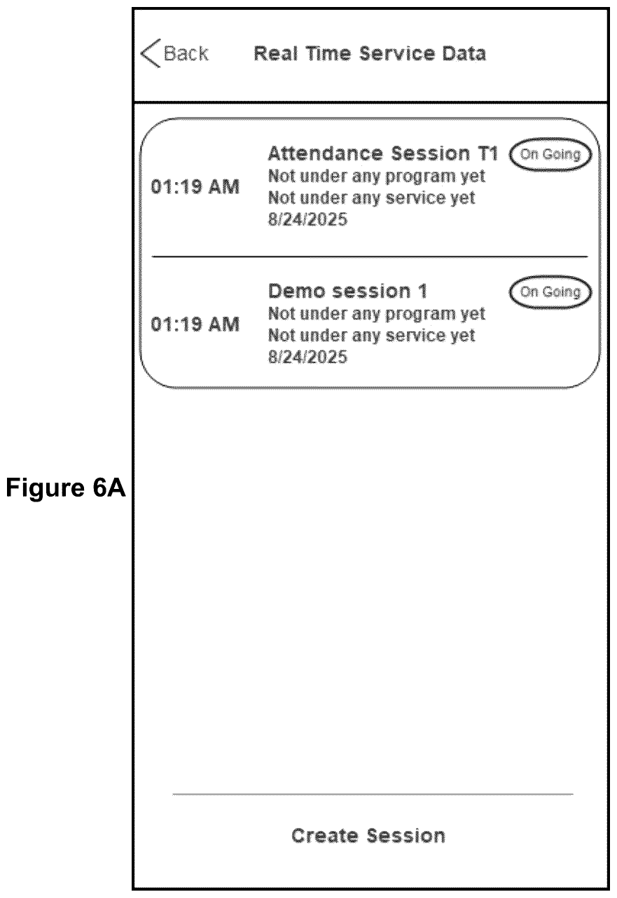

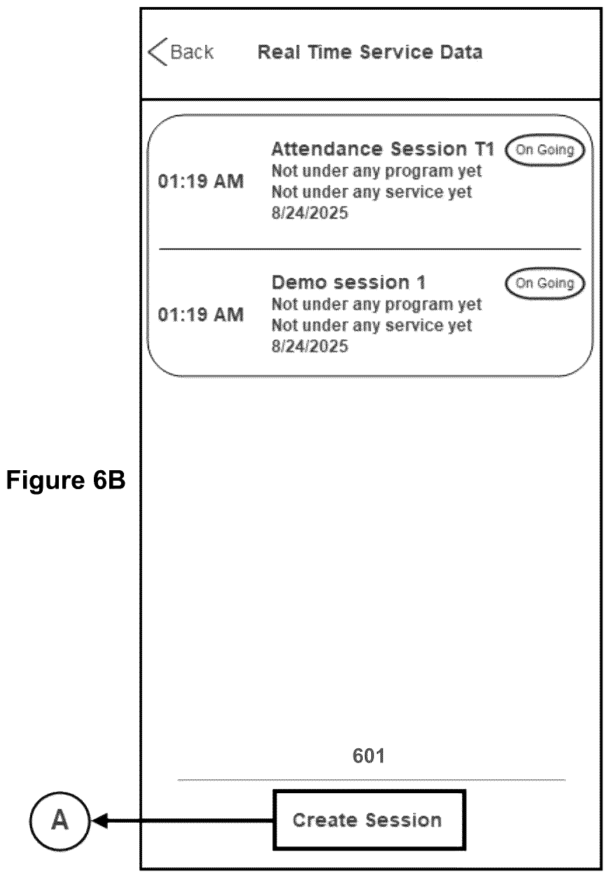

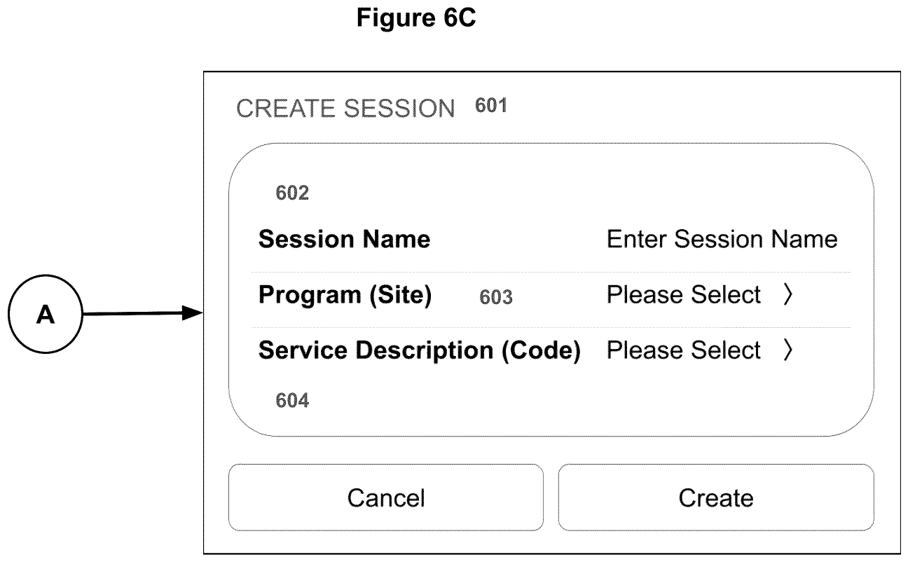

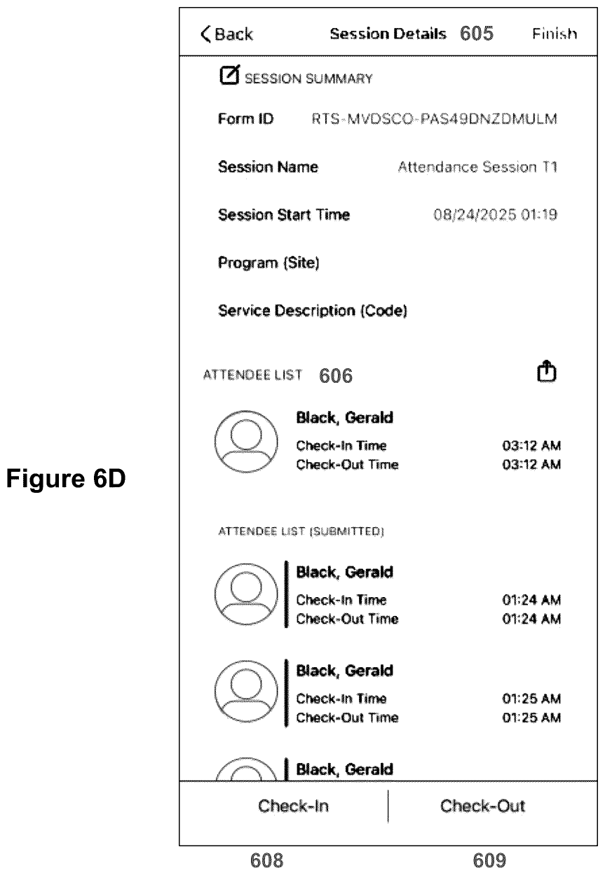

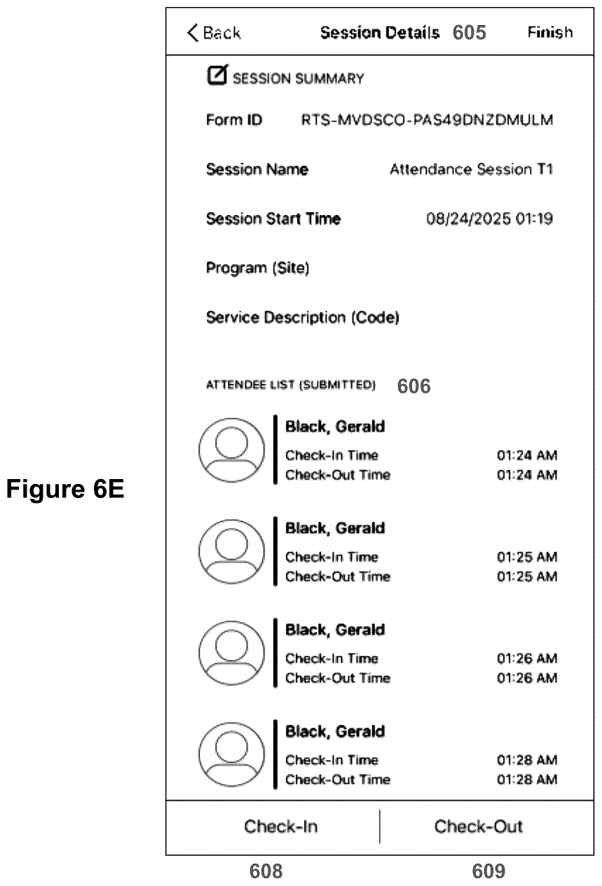

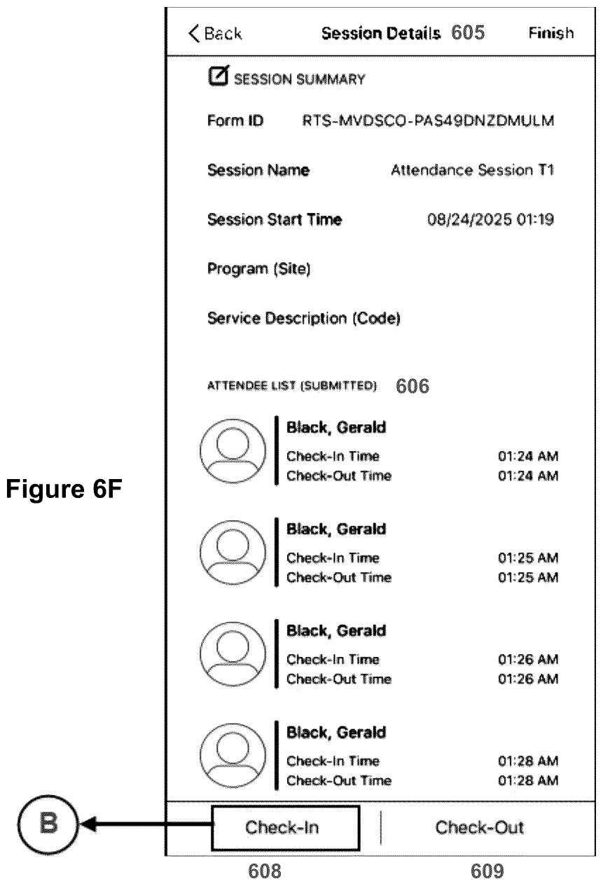

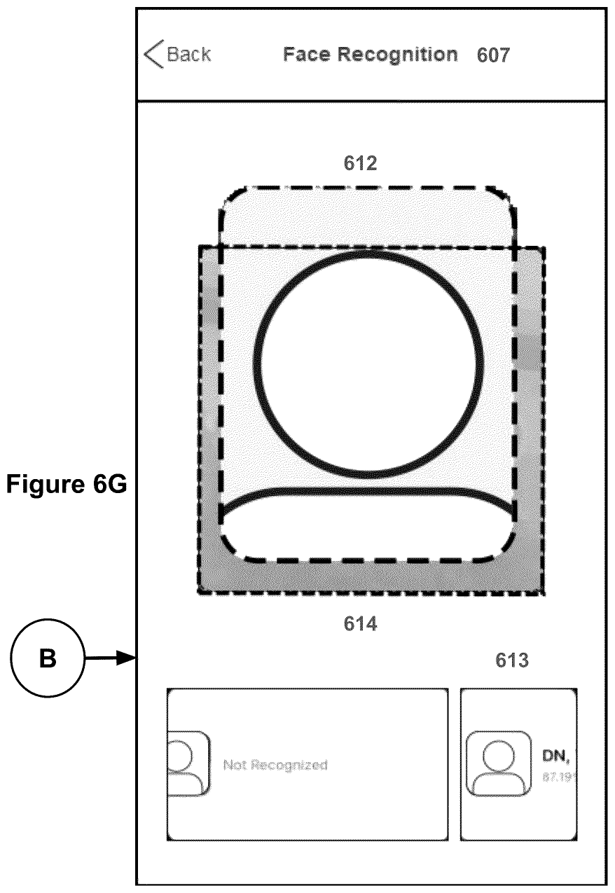

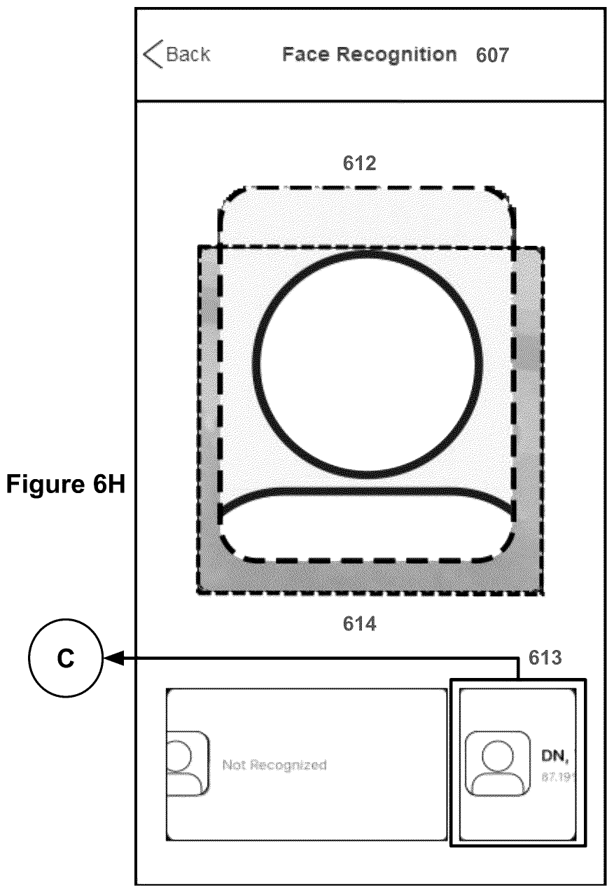

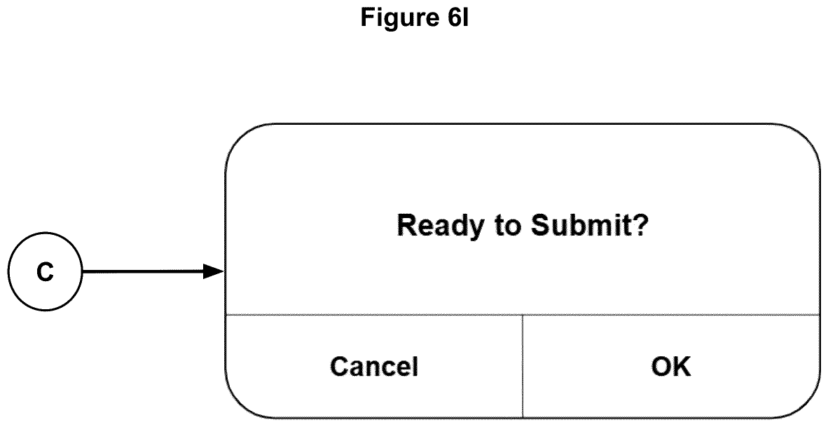

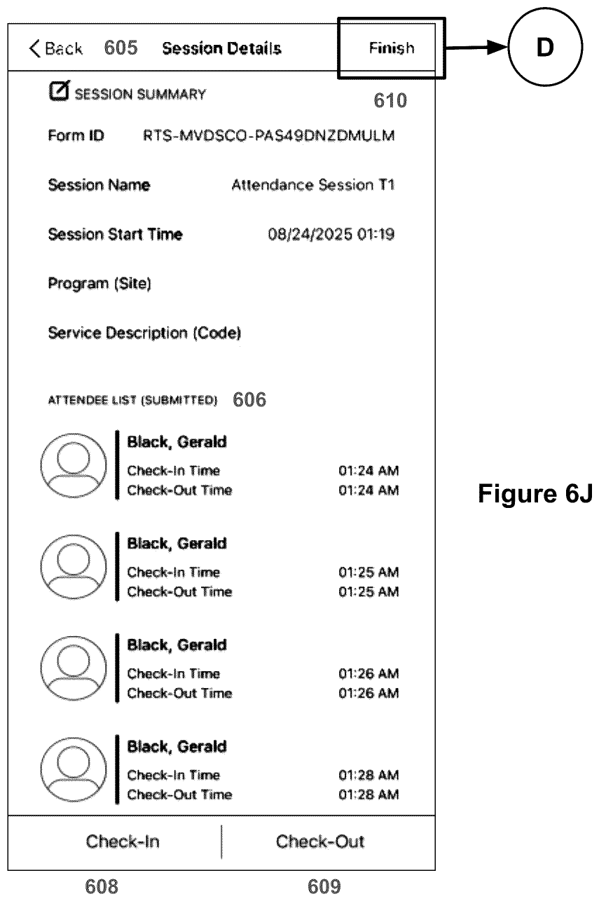

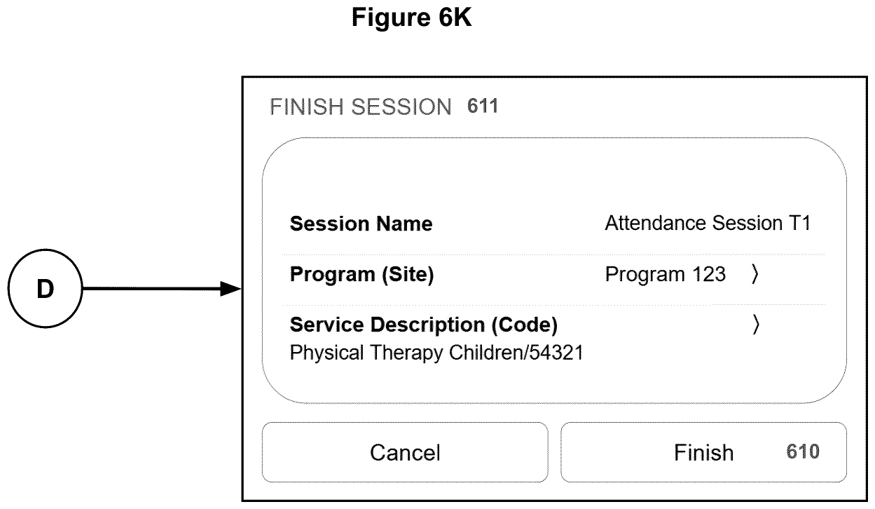

FIG. 6A depicts the Session List for the Real Time Service Data module.

FIG. 6B depicts a connector box labeled ‘A’ which highlights the Create Session 601 button.

FIG. 6C, which follows the flow from connector ‘A’ in the previous FIG. 6B, depicts that the system configured to three fields need to be filled in order for a user to Create Session 601. The first field, Session Name 602 is a required field and the fields Program (Site) 603 and Service Description (Code) 604 may be added as per requirement.

FIG. 6D illustrates the Session Summary 605. This summary comprises attendees who have either checked-in or out of the session.

FIG. 6E illustrates the Session Summary 605 after the submission of the Attendee List 606.

FIG. 6F depicts a connector box labeled ‘B’ which highlights the Check-In 608 button.

As shown in FIG. 6G, which follows the flow from connector ‘B’ in the previous FIG. 6F, the system comprises Face Recognition 607 to Check-In 608 and Check-Out 609 from a session. A user may check-in and check-out one or more attendees using Face Recognition 607 at a time. It should be noted that in order for the Face Recognition 607 to work during check-in 608 and check-out 609, an attendee needs to be previously registered in the system.

FIG. 6H illustrates a connector box labeled ‘C’ which highlights the registration of Face Recognition 607.

FIG. 6I illustrates the pop-up for data submission, which follows the flow from connector ‘C’ in the previous FIG. 6H. Upon completing the Face Recognition 607 process, a user may submit data for one or more attendees as required.

FIG. 6J depicts a connector box labeled ‘D’which highlights the Finish 610 button.

FIG. 6K, which follows the flow from connector ‘D’ in the previous FIG. 6J, illustrates the process to Finish Session 611. A user needs to click on the Finish 610 button at the top in order to get the pop-up as shown in FIG. 6K. The user then clicks on the Finish 610 button at the bottom of the page to end the session.

Real Time Service Data Capture: The following text describes the “Real Time Service Data” feature.

Overview: The Real Time Service Data feature involves collecting data from the Therap Mobile App, buffering the data, displaying it on a web application dashboard, and further processing the data for purposes such as sanitation and generating attendance records. This feature preferably includes four key components, described below.

Data Collection from Therap Mobile App component: Essential Data Points includes Facial Image, Mapped Individual—DB ID, Check In/Out Time, Location (Latitude and longitude), Session Name (Optional), Service Name (Optional), Program Name (Optional).

Data Buffering component: Storing real time data for post processing.

Dashboard Display in Web Application component: Visualizing real-time data on a web interface.

Data Processing component: Sanitizing and processing data for further analysis and use, including attendance tracking. Implementation, App Prototype.

The implementation of each of these components is described below.

Data Collection From Mobile: In the mobile data collection process, a session must first be created with a mandatory session name. Before the session ends, users may optionally add the program name and service name as session details. Once the session starts, there are two options: Check In or Check Out. When a user selects Check In, the camera opens to capture real-time data through facial recognition. Later, when the person leaves, Check Out is clicked to record the checkout time. All captured data is then transmitted to the web application buffer for further processing.

Dashboard Display and Data Processing Workflow (Web): To begin, open the screen; it displays a session date search box while the dashboard remains empty. Next, enter a session date and click Search. This loads the records for that date or display “No Data Available” if none exist. Afterward, select a Program and a Description Code, which enables the Choose Service button. Clicking this button opens a popup that lists the related services for the selected Program, Description, Individual, and Date. From there, choose a Service along with an Attendance Type. The selected row updates with the selected choice, and the Generate button becomes active. When Generate Data is clicked, attendance is created for that record. If successful, the system displays a Generated Attendance ID List with the corresponding Service IDs. In the case of an error, it shows “Failed Processing.”

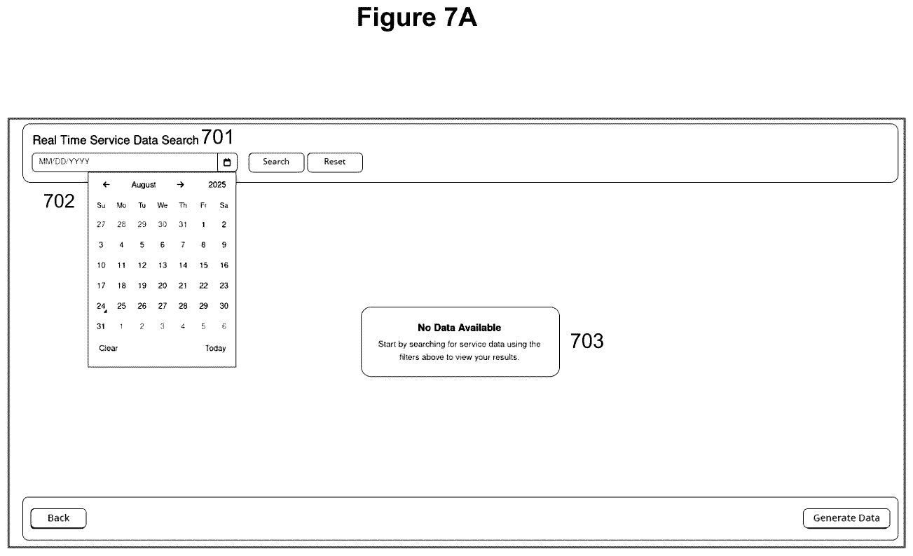

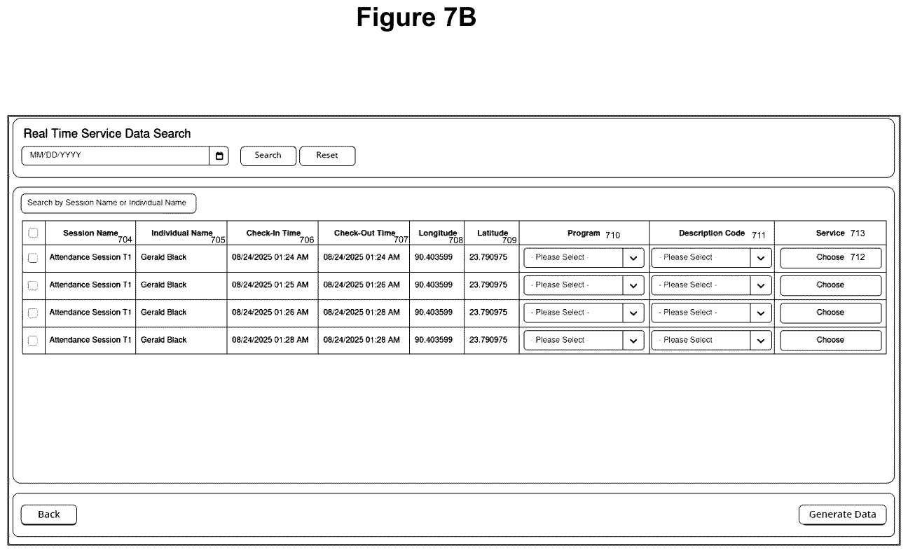

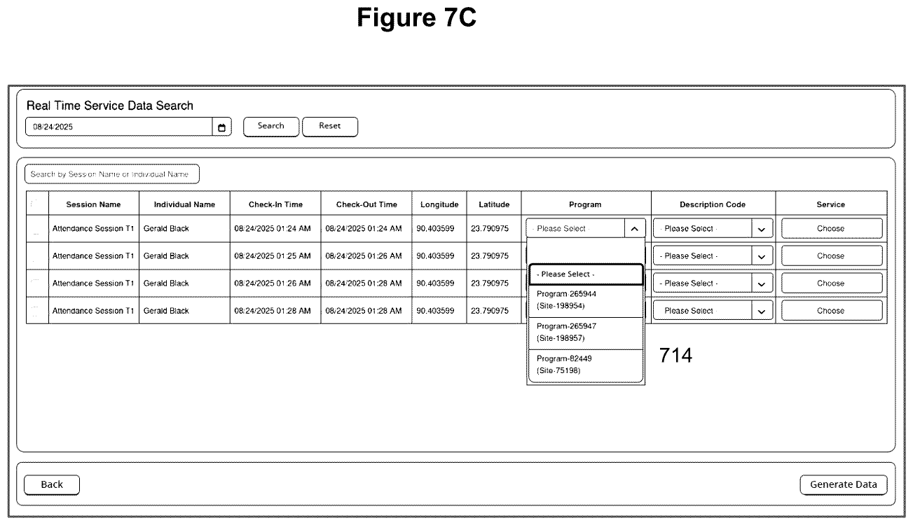

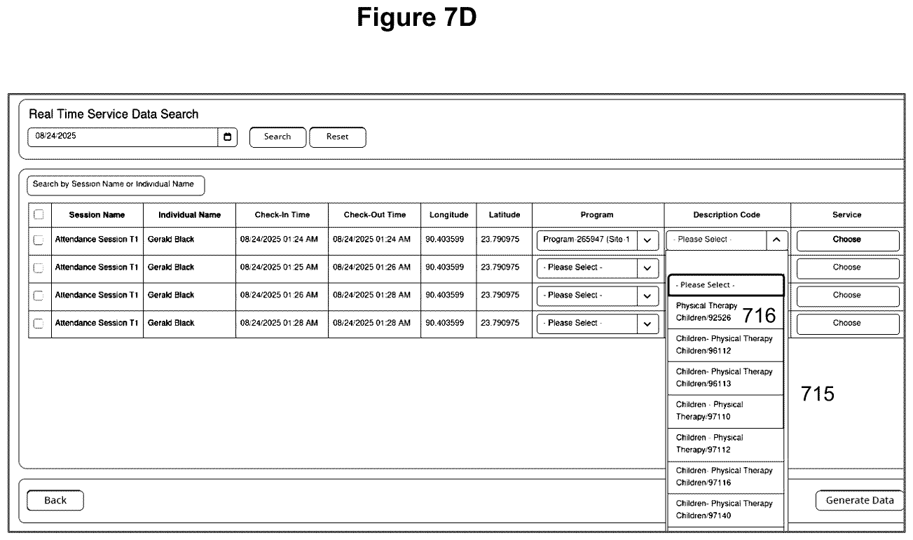

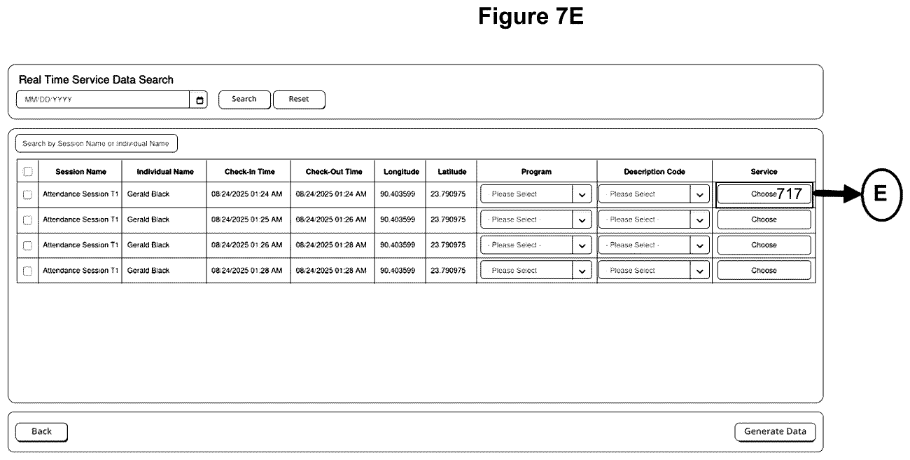

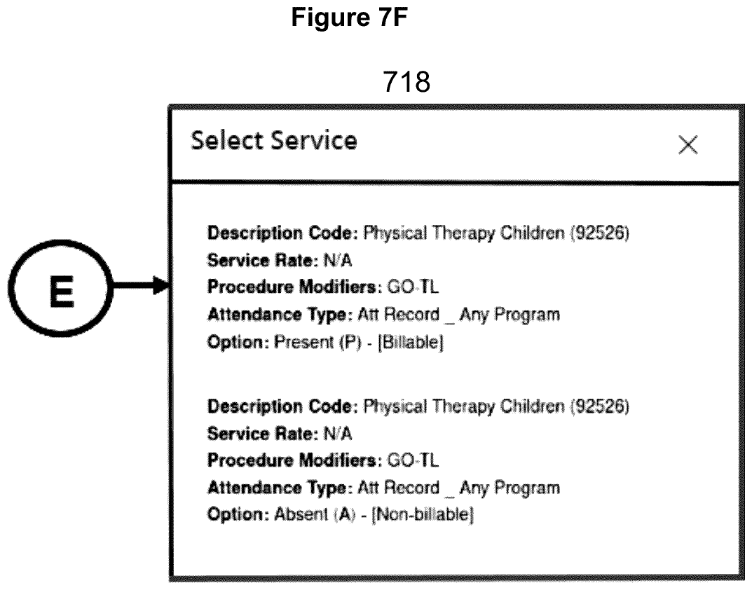

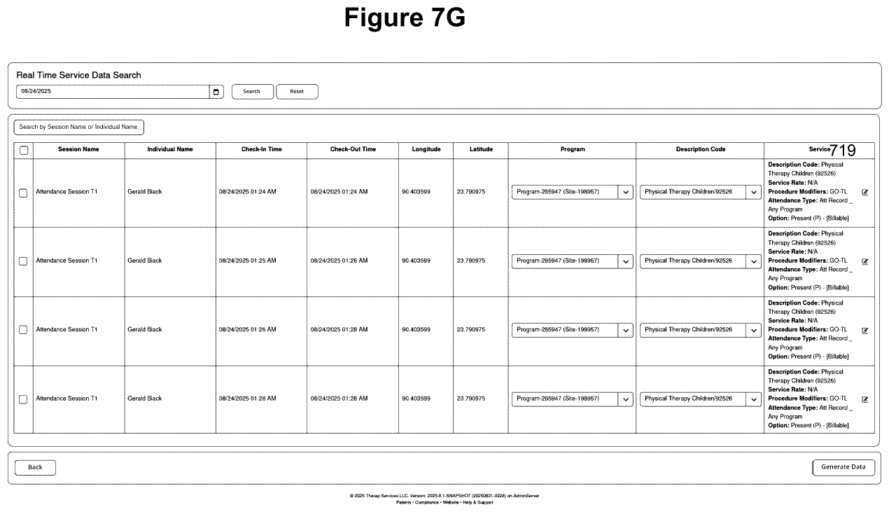

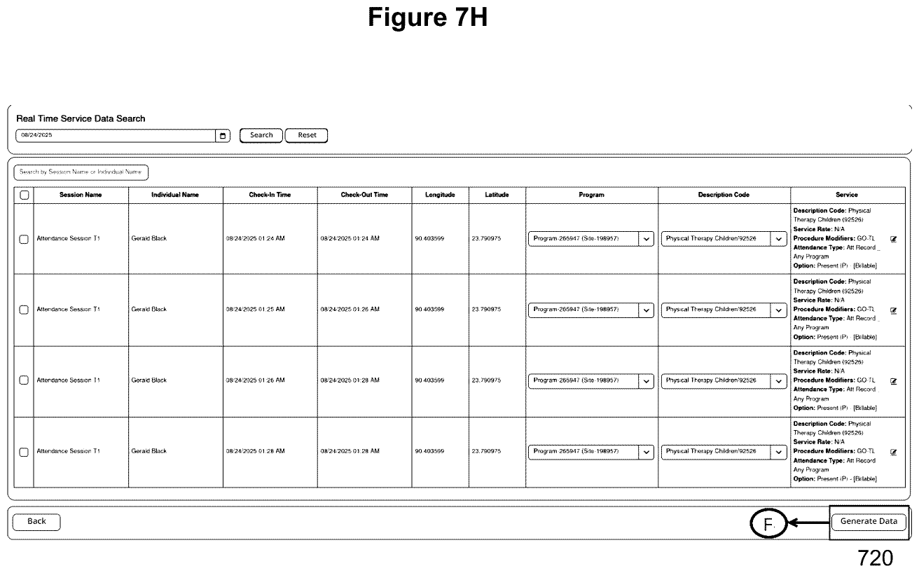

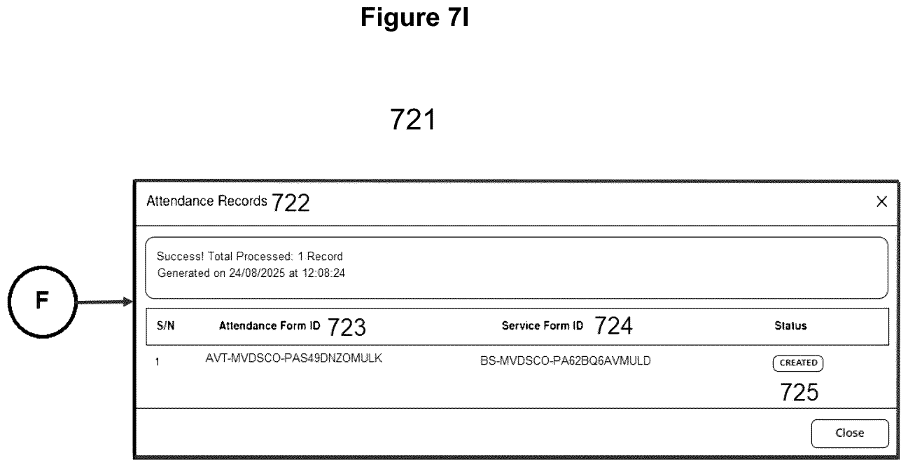

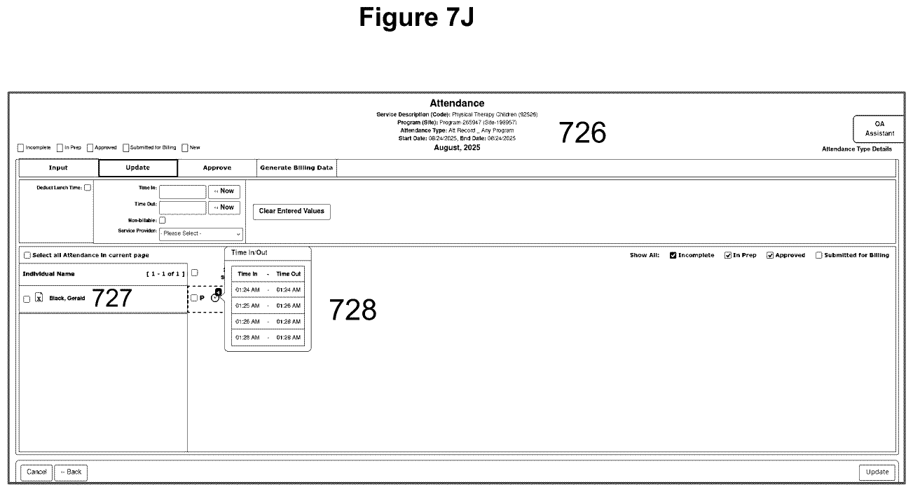

FIG. 7A depicts the initial user interface for the Real Time Service Data Search 701 feature. The user is presented with a date selection field labeled ‘MM/DD/YYYY’ 702 and a calendar widget to select a specific date. The main content area displays a ‘No Data Available’ 703 message, indicating that a search must be performed first.

FIG. 7B depicts the interface after the user has selected a date and executed a search. A data grid is now populated with multiple rows, each representing a real-time service data entry for an individual. The grid includes columns for ‘Session Name’ 704, ‘Individual Name’ 705, ‘Check-in Time’ 706, ‘Check-Out Time’ 707, ‘Longitude 708’, and ‘Latitude’ 709. The ‘Program’ 710 and ‘Description Code’ 711 columns contain dropdown menus, and a ‘Choose’ 712 button is present in the ‘Service’ 713 column for each entry.

FIG. 7C shows the user interacting with the ‘Program’ dropdown 714 menu for one of the data entries. A list of available programs is displayed for the user to select from.

FIG. 7D illustrates the user interacting with the ‘Description Code’ 715 dropdown menu after a program has been selected. A list of relevant description codes, such as ‘Physical Therapy Children/92526’ 716, is presented to the user.

FIG. 7E shows the data grid where the ‘Program’ and ‘Description Code’ have been selected for the entries. A connector box labeled ‘E’ highlights that the selection of these two fields initiates the next step of the workflow by selecting the Choose 717 button.

FIG. 7F, which follows the flow from connector ‘E’ in the previous FIG. 7E, illustrates the ‘Select Service’ 718 pop-up window. This window allows the user to specify the details for the selected service, providing options such as ‘Present (P)—[Billable]’ and ‘Absent (A)—[Non-billable]’ for the ‘Attendance Type.’

FIG. 7G depicts the state of the data grid after the user has made a selection in the ‘Select Service’ 718 pop-up from FIG. 7F. The ‘Service’ 719 column for each row is now populated with the detailed service information, including the Description Code, Service Rate, Procedure Modifiers, and the chosen Attendance Type option.

FIG. 7H depicts a connector box labeled ‘F’ that highlights the ‘Generate Data’ 720 button, indicating the next action is to finalize and create the attendance records.

FIG. 7I, following the flow from connector ‘F’ in the previous FIG. 7H, shows the success confirmation pop-up 721 that appears after the ‘Generate Data’ 720 button is clicked. The pop-up, titled ‘Attendance Records’ 722, confirms the successful processing of the records and displays the newly created ‘Attendance Form ID’ 723 and ‘Service Form ID’ 724 with a ‘CREATED’ 725 status.

FIG. 7J illustrates the final outcome of the process, showing the main ‘Attendance’ 726 dashboard. The newly generated attendance record for the individual, ‘Black, Gerald’ 727, is now visible in the attendance grid, confirming that the data has been successfully converted and stored as a formal attendance entry. A ‘Time In/Out’ 728 tooltip shows the specific session times that were captured.

It will be understood by those of ordinary skill in the art that various changes may be made and equivalents may be substituted for elements without departing from the scope of the invention. In addition, many modifications may be made to adapt a particular feature or material to the teachings of the invention without departing from the scope thereof. Therefore, it is intended that the invention not be limited to the particular embodiments disclosed, but that the invention will include all embodiments falling within the scope of the claims.

1. An improvement to the way that computer systems operate to electronically record the presence of an individual at a location, including individuals under care by a caregiver, the improvement comprising a HIPAA-compliant method of receiving and electronically recording personal identification information data relating to the physical appearance of at least one individual, comparing the records to previously-stored records to determine a match, and creating and storing an electronic attendance record indicating the presence of the individual at the location, the method comprising the steps of:
a. providing a database of visual stored personal identification information data relating to the physical appearance of at least one individual;
b. providing a database of stored hashes of said data;
c. providing a first sensor for capturing images at a first point, said first point within the location;
d. providing a computer system connected to said first sensor and said hash database;
e. providing an attendance database for storing electronic records relating to the presence of the individual in said point;
f. capturing, by said first sensor, a first image of the individual as an electronic record;
g. creating a first electronic image hash of said first electronic record;
h. comparing, by said computer system, said first electronic image record hash to said stored hashes to determine a match; and
i. creating, by said computer system, if said first electronic image record hash matches one of said stored hashes, a first electronic attendance record indicating that the individual is present at said first point; and
j. storing, in said attendance database, said first electronic attendance record.

2. The method of claim 1 wherein the individual is an individual under care, and further including performing, by the computer system, the steps of:
a. providing a database storing at least one authorization profile associated with a caregiver, wherein the caregiver is associated with one or more roles and one or more caseloads and said caseloads include access privilege information for the individual, wherein said access privilege information included in said caseload includes the identities of individuals to which the caregiver has access;
b. comparing the identity of the individual to the caregiver's authorization profile information, including comparing said identity of the individual to the caregiver's caseload; and
c. providing access to said first electronic attendance record to the caregiver if said identity of the individual is stored in the caregiver's caseload.

3. The method of claim 1 wherein:
a. the individual is a caregiver and an individual under care is located at said location; and
b. said first electronic attendance record indicates, at least in part, that said caregiver is at said location for providing service to the individual under care.

4. The method of claim 1 wherein said first sensor is configured to capture an image of the individual entering said location at said first point, and wherein said electronic attendance record indicates, at least in part, that said individual has entered said location.

5. The method of claim 4 further comprising:
a. providing a second sensor for capturing images at a second point, said second point within said location, and wherein said second sensor is configured to capture an image of the individual exiting said location at said second point;
b. capturing, by said second sensor, a second image of the individual as an electronic record;
c. creating a second electronic image hash of said second electronic record;
d. comparing, by said computer system, said second electronic image record hash to said stored hashes to determine a match; and
e. creating, by said computer system, if said second electronic image record hash matches one of said stored hashes, a second electronic attendance record indicating that the individual is present at said second point; and
f. storing in said attendance database, said second electronic attendance record.

6. The method of claim 5 wherein said first electronic attendance record further includes one or more of a date and time corresponding to said first image, and said second electronic attendance record further includes one or more of a date and time corresponding to said second image, further comprising the steps of:
a. creating, by said computer system, a session electronic attendance record based on said one or more of said date and time of said first and second attendance records; and
b. storing, in said attendance database, said session electronic attendance record.

7. The method of claim 6 wherein said session electronic attendance record includes service information.

8. The method of claim 6 wherein said session electronic attendance record includes program information.

9. The method of claim 1 wherein said first sensor performs said first electronic image hash creation step, and further including the step of transmitting, by said first electronic sensor to said computer system, said first electronic image hash.

10. The method of claim 1 wherein said first sensor is the camera of a mobile phone.

11. The method of claim 3 wherein said first attendance record includes data identifying:
a. the type of service performed;
b. the individual receiving the service;
c. the date of the service;
d. the location of service delivery;
e. the individual providing the service; and
f. the time the service begins and ends.

12. The method of claim 11 wherein said first attendance record is an electronic visit verification.

13. The method of claim 1 wherein said hashes of said data and said first electronic image hash are each machine learning embeddings.

14. The method of claim 1 wherein said hash database is one or more of a machine learning vector database and a feature vector database.

15. The method of claim 1 wherein said comparing step is a machine learning nearest neighbor search.

16. The method of claim 15 wherein:
a. said nearest neighbor search returns a probability of a match of said first electronic image record hash and one of said stored hashes; and
b. said first electronic image record hash matches one of said stored hashes if said probability exceeds a predetermined threshold for determining said match.

17. An improvement to computer systems that operate to electronically record the presence of an individual at a location, including individuals under care by a caregiver, the improvement comprising a HIPAA-compliant computer system for receiving and electronically recording personal identification information data relating to the physical appearance of at least one individual, comparing the records to previously-stored records to determine a match, and creating and storing an electronic attendance record indicating the presence of the individual at the location, the system comprising:
a. a database of visual stored personal identification information data relating to the physical appearance of at least one individual;
b. a database of stored hashes of said data;
c. a first sensor for capturing images at a first point, said first point within the location;
d. a computer system connected to said first sensor and said hash database;
e. an attendance database for storing electronic records relating to the presence of the individual in said point;
f. said first sensor configured to capture a first image of the individual as an electronic record; and
g. one of said second sensor and said computer system is configured to create a first electronic image hash of said first electronic record;
h. said computer system configured to:
i. compare said first electronic image record hash to said stored hashes to determine a match; and
ii. create, if said first electronic image record hash matches one of said stored hashes, a first electronic attendance record indicating that the individual is present at said first point; and
iii. storing said first electronic attendance record in said attendance database.

18. The system of claim 17 wherein the individual is an individual under care, and further comprising:
a. a database storing at least one authorization profile associated with a caregiver, wherein the caregiver is associated with one or more roles and one or more caseloads and said caseloads include access privilege information for the individual, wherein said access privilege information included in said caseload includes the identities of individuals to which the caregiver has access; and
b. wherein said computer system is further configured to:
i. compare the identity of the individual to the caregiver's authorization profile information, including to compare said identity of the individual to the caregiver's caseload; and
ii. provide access to said first electronic attendance record to the caregiver if said identity of the individual is stored in the caregiver's caseload.

19. The system of claim 17 wherein:
a. the individual is a caregiver and an individual under care is located at said location; and
b. said first electronic attendance record indicates, at least in part, that said caregiver is at said location for providing service to the individual under care.

20. The system of claim 17 wherein said first sensor is configured to capture an image of the individual entering said location at said first point, and wherein said electronic attendance record indicates, at least in part, that said individual has entered said location.

21. The system of claim 20 further comprising:
a. a second sensor for capturing images at a second point, said second point within said location, and wherein said second sensor is configured to capture a second image of the individual exiting said location at said second point as an electronic record;
b. one of said second sensor and said computer system is further configured to create a second electronic image hash of said second electronic record; and
c. said computer system is further configured to:
i. compare said second electronic image record hash to said stored hashes to determine a match;
ii. create, if said second electronic image record hash matches one of said stored hashes, a second electronic attendance record indicating that the individual is present at said second point; and
iii. store said second electronic attendance record in said attendance database,.

22. The system of claim 21 wherein said first electronic attendance record further includes one or more of a date and time corresponding to said first image, and said second electronic attendance record further includes one or more of a date and time corresponding to said second image, wherein said computer system is further configured to:
a. create a session electronic attendance record based on said one or more of said date and time of said first and second attendance records; and
b. Store said session electronic attendance record in said attendance database,.

23. The system of claim 22 wherein said session electronic attendance record includes service information.

24. The system of claim 22 wherein said session electronic attendance record includes program information.

25. The system of claim 17 wherein said first sensor is configured to create said first electronic image hash creation step, and further configured to transmit said first electronic image hash to said computer system.

26. The system of claim 17 wherein said first sensor is the camera of a mobile phone.

27. The system of claim 19 wherein said first attendance record includes data identifying:
a. the type of service performed;
b. the individual receiving the service;
c. the date of the service;
d. the location of service delivery;
e. the individual providing the service; and
f. the time the service begins and ends.

28. The system of claim 27 wherein said first attendance record is an electronic visit verification.

29. The system of claim 17 wherein said hashes of said data and said first electronic image hash are each machine learning embeddings.

30. The system of claim 17 wherein said hash database is one or more of a machine learning vector database and a feature vector database.

31. The system of claim 17 wherein said computer system is further configured to compare said second electronic image record hash to said stored hashes to determine a match using a machine learning nearest neighbor search.

32. The system of claim 31 wherein:
a. said nearest neighbor search returns a probability of a match of said first electronic image record hash and one of said stored hashes; and
b. said first electronic image record hash matches one of said stored hashes if said probability exceeds a predetermined threshold for determining said match.
